(19) United States
(12) Patent Application Publication
(10) Pub. No.: US 2008/0103198 A1
(43) Pub. Date: May 1, 2008

(54) METHODS FOR TREATMENT OF COCHLEAR AND VESTIBULAR DISORDERS

(76) Inventor: Magali HAAS, Corinth, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/870,511

(22) Filed: Oct. 11, 2007

Related U.S. Application Data
(60) Provisional application No. 60/863,144, filed on Oct. 27, 2006.

(51) Int. Cl. A61K 31/27; A61P 27/16

(52) U.S. Cl. 514/483; 514/489

(57) ABSTRACT

This invention is directed to methods for providing otoprotection comprising administering to a subject in need thereof a therapeutically effective amount of a compound selected from the group consisting of Formula (I) and Formula (II), or a pharmaceutically acceptable salt or ester thereof:

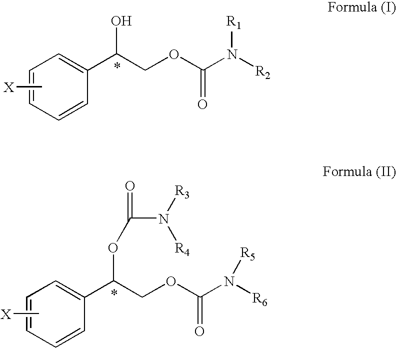

wherein phenyl is substituted at X with one to five halogen atoms selected from the group consisting of fluorine, chlorine, bromine and iodine; and, R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

This application claims the benefit under 35 U.S.C. 119(e) of U.S. Provisional application Ser. No. 60/863,144 filed Oct. 27, 2006. The complete disclosure of the aforementioned related U.S. patent application is hereby incorporated herein by reference for all purposes.

### BACKGROUND OF THE INVENTION

1. Field of the Invention

The present invention relates generally to the fields of pharmacology and neurology and to methods of protecting the cells of a mammalian inner ear and auditory nerve, including the cochlea and vestibular system, from damage or degeneration. More specifically, this invention provides methods for the use of certain carbamate compounds for treatment of cochlear and vestibular disorders.

2. Description of the Related Art

Loss of hearing and balance impairments are serious handicaps that affect millions of people. Hearing impairments can be attributed to a wide variety of causes, including infections, mechanical injury, exposure to loud sounds, aging, and chemical-induced ototoxicity that damages neurons and/or hair cells of the peripheral auditory system.

The peripheral auditory system consists of auditory receptors, hair cells in the organ of Corti, and primary auditory neurons, the spiral ganglion neurons in the cochlea. Spiral ganglion neurons (“SGN”) are primary afferent auditory neurons that deliver signals from the peripheral auditory receptors, the hair cells in the organ of Corti, to the brain through the cochlear nerve. The eighth nerve connects the primary auditory neurons in the spiral ganglia to the brain stem. The eight nerve also connects vestibular ganglion neurons (“VGN”), which are primary afferent sensory neurons responsible for balance and which deliver signals from the utricle, saccule and ampullae of the inner ear to the brain, to the brainstem. Destruction of primary afferent neurons in the spiral ganglia and hair cells has been attributed as a major cause of hearing impairments. Damage to the peripheral auditory system is responsible for a majority of hearing deficits (Dublin, 1976; Rybak, 1986; Lim, 1986; Pryor, 1994).

Hearing loss or impairment is a common cause of disability in humans. Impairment anywhere along the auditory pathway from the external auditory canal to the central nervous system may result in hearing loss or balance impairment. Auditory apparatus can be divided into the external and middle ear, inner ear and auditory nerve and central auditory pathways. While having some variations from species to species, the general characterization is common for all mammals. Auditory stimuli are mechanically transmitted through the external auditory canal, tympanic membrane, and ossicular chain to the inner ear.

The middle ear and mastoid process are normally filled with air. Disorders of the external and middle ear usually produce a conductive hearing loss by interfering with this mechanical transmission. Common causes of a conductive hearing loss include obstruction of the external auditory canal, as can be caused by aural atresia or cerumen; thickening or perforation of the tympanic membrane, as can be caused by trauma or infection; fixation or resorption of the components of the ossicular chain; and obstruction of the Eustachian tube, resulting in a fluid-filled middle-ear space.

Auditory information is transduced from a mechanical signal to a neurally conducted electrical impulse by the action of neuro-epithelial cells (hair cells) and SGN in the inner ear. All central fibers of SGN form synapses in the cochlear nucleus of the pontine brain stem. The auditory projections from the cochlear nucleus are bilateral, with major nuclei located in the inferior colliculus, medial geniculate body of the thalamus, and auditory cortex of the temporal lobe. The number of neurons involved in hearing increases dramatically from the cochlea to the auditory brain stem and the auditory cortex. All auditory information is transduced by a limited number of hair cells, which are the sensory receptors of the inner ear, of which the so-called inner hair cells, numbering a comparative few, are critically important, since they form synapses with approximately 90 percent of the primary auditory neurons.

By comparison, at the level of the cochlear nucleus, the number of neural elements involved is measured in the hundreds of thousands. Thus, damage to or disease of a relatively few cells in the auditory periphery can lead to substantial hearing loss or balance impairment. Hence, many causes of sensorineural loss can be ascribed to lesions in the inner ear. This hearing loss and balance impairment can be progressive. In addition, the hearing becomes significantly less acute because of changes in the anatomy of the ear as the animal ages.

During embryogenesis, the vestibular ganglion, spiral ganglion, and the otic vesicle are derived from the same neurogenic ectoderm, the otic placode. The vestibular and auditory systems thus share many characteristics including peripheral neuronal innervations of hair cells and central projections to the brainstem nuclei. Both of these systems are sensitive to ototoxins that include therapeutic drugs, antineoplastic agents, contaminants in foods or medicines, and environmental and industrial pollutants. Ototoxic drugs include the widely used chemotherapeutic agent cisplatin and its analogs (Fleischman et al., 1975; Stadnicki et al., 1975; Nakai et al., 1982; Berggren et al., 1990; Dublin, 1976; Hood and Berlin, 1986), commonly used aminoglycoside antibiotics, e.g. gentamicin, for the treatment of infections caused by Gram-negative bacteria, (Sera et al., 1987; Hinojosa and Lerner, 1987; Bareggi et al., 1990), quinine and its analogs, salicylate and its analogs, and loop-diuretics.

The toxic effects of these drugs on auditory cells and spiral ganglion neurons are often the limiting factor for their therapeutic usefulness. For example, antibacterial aminoglycosides such as gentamicins, streptomycins, kanamycins, tobramycins, and the like are known to have serious toxicity, particularly ototoxicity and nephrotoxicity, which reduce the usefulness of such antimicrobial agents (see Goodman and Gilman's The Pharmacological Basis of Therapeutics, 6th ed., A. Goodman Gilman et al., eds; Macmillan Publishing Co., Inc., New York, pp. 1169-71 (1980) or most recent edition). Aminoglycoside antibiotics are generally utilized as broad spectrum antimicrobials effective against, for example, gram-positive, gram-negative and acid-fast bacteria.

The aminoglycosides are used primarily to treat infections caused by gram-negative bacteria and, for instance, in combination with penicillins for the synergistic effects. As implied by the generic name for the family, all the aminoglycoside antibiotics contain aminosugars in glycosidic linkage. Otitis media is a term used to describe infections of the middle ear, which infections are very common, particularly in children. Typically antibiotics are systemically administered for infections of the middle ear, e.g., in a responsive or prophylactic manner. Systemic administration of antibiotics to combat middle ear infection generally results in a prolonged lag time to achieve therapeutic levels in the middle ear, and requires high initial doses in order to achieve such levels. These drawbacks complicate the ability to obtain therapeutic levels and may preclude the use of some antibiotics altogether.

Systemic administration is most often effective when the infection has reached advanced stages, but at this point permanent damage may already have been done to the middle and inner ear structure. Clearly, ototoxicity is a dose-limiting side-effect of antibiotic administration. For example, nearly 75% of patients given 2 grams of streptomycin daily for 60 to 120 days displayed some vestibular impairment, whereas at 1 gram per day, the incidence decreased to 25%. Hearing loss can be also observed with some 4 to 15% of patients receiving 1 gram streptomycin per day for greater than 1 week developing measurable hearing loss. This hearing loss may progress and can lead to complete permanent deafness if treatment continues.

In addition, ototoxicity is also a serious dose-limiting side-effect for cisplatin, a platinum coordination complex, that has proven effective on a variety of human cancers including testicular, ovarian, bladder, and head and neck cancer. Cisplatin damages auditory and vestibular systems (Fleischman et al., 1975; Stadnicki et al., 1975; Nakai et al., 1982; Carenza et al., 1986; Sera et al., 1987; Bareggi et al., 1990). Salicylates, such as aspirin, are the most commonly used therapeutic drugs for their anti-inflammatory, analgesic, anti-pyretic and anti-thrombotic effects. Unfortunately, they have ototoxic side effects. They often lead to tinnitus (“ringing in the ears”) and temporary hearing loss (Myers and Bernstein, 1965). However, if the drug is used at high doses for a prolonged time, the hearing impairment can become persistent and irreversible, as reported clinically (Jarvis, 1966).

In addition to ototoxic drugs a wide variety of diseases and degenerative conditions adversely affect the inner ear hair cells and associated neurons. For example, immune mediated ear disorders, such as immune-mediated cochlear or vestibular disorders (IMCVD), continue to present a management challenge to the otolaryngologist. These disorders represent a syndrome of sensorineural hearing loss, often associated with vertigo, tinnitus, and aural fullness believed to be due to an autoimmune mechanism. The sequelae of IMCVDs include devastating disabilities, such as profound deafness and serious vestibular dysfunction. Immunosuppressive drugs like cyclophosphomide and anti-rheumatic agents like methotrexate are employed for IMCVD, but are associated with variable efficacy, slow onset of effects, and sometimes serious toxicity. (Rahman, M U et al. Curr. Opin. Rheumatol., May; 13(3):184-9, (2001))

The terms “immune-mediated ear disorder” or “immune-mediated ear disease” refer to impairment of ear function that are brought about by an immune-based mechanism, such as an autoimmune or inflammatory response. Any portion of the ear may be affected, but the inner ear is most often compromised. Immune-mediated ear disorders include, without limitation, immune-mediated cochlear or vestibular disorders (IMCVD), immune-mediated Meniere's disease, autoimmune ear disease (AIED), Cogan's Syndrome, and Wegener's granulomatosis. Symptoms related to immune-mediated ear disorders include, hearing impairment (including full or partial hearing loss in one or both ears), vertigo, tinnitus, fullness in the ear, otalgia, otorrhea/chronicotitis media, and TM perforation.

Additional disorders affecting the inner ear include Meniere's disease. A typical attack of Meniere's disease is preceded by fullness in one ear, hearing fluctuations or tinnitus may also precede the attack. The full episode generally involves severe vertigo, imbalance, nausea and vomiting and may last for two to four hours. Meniere's disease may cause sudden fall that occur with out warning caused by a sudden activation of vestibular reflexes. The disease is not fatal but can be extremely disabling and can cause progressive hearing loss. Meniere's disease is fairly common occurring in 0.2% of the population or some 600,000 people in the US alone. The cause is not known but is believed to be due to viral and/or immunologic possesses affecting the inner ear but the end result is often the degeneration of the cochlear and/or vestibular system.

Tinnitus is a common is a ringing, hissing or roaring sound heard by the patient that is not caused by any sound in the environment. Tinnitus is extremely common, affecting some 36 million Americans with about 6% of the general population having severe symptoms. Tinnitus is a symptom rather than a specific disease and has many causes including mechanical injury from exposure to loud noise, degenerative diseases including Meniere's disease but most tinnitus results from damage to the cochlea or the vestibular nerve. Many common drugs can also cause tinnitus including aspirin, NSAIDS, loop diuretics such as Lasix, antibiotics, quinine and many kind osf chemotherapy agents such as cis platinum as described above.

Accordingly, there exists a need for means to prevent, reduce or treat the incidence and/or severity of inner ear disorders and hearing impairments involving inner ear tissue, particularly inner ear hair cells, and the associated auditory nerves. Of particular interest are those conditions arising as an unwanted side-effect of ototoxic therapeutic drugs including cisplatin and its analogs, aminoglycoside antibiotics, salicylate and its analogs, or loop diuretics and degenerative diseases involving inner ear hair cells and associated neurons including ischemia. In addition, there exits a need for methods that will allow higher and thus more effective dosing with these ototoxicity-inducing pharmaceutical drugs, while concomitantly preventing or reducing ototoxic effects caused by these drugs. What is needed is a method that provides a safe, effective, and prolonged means for prophylactic or curative treatment of hearing impairments related to inner ear tissue damage, loss, or degeneration, particularly ototoxin-induced and degenerative diseases involving inner ear hair cells and associated neurons.

Therefore a serious need continues to exist for a method of treatment hearing and balance impairments and other conditions associated with damage to hair cells and/or their supporting cells including the eighth nerve. The present invention provides compositions and methods to protect these regions from the effects of toxic drugs and degenerative disorders of many kinds.

Injuries or trauma of various kinds to the cochlea or the vestibular system or the auditory or vestibular nerves including the primary auditory receptors, the hair cells in the Organ of Corti, and the primary auditory neurons and the spiral ganglion neurons in the cochlea, the neurons of the eight nerve and the vestibular ganglion neurons, can produce profound and long-lasting hearing loss or balance problems or incapacitating symptoms such as vertigo, nausea and vomiting. Some of these effects are caused by the progressive death of neurons or other related cells of the inner ear, i.e., neurodegeneration or neuronal degeneration.

The mechanisms of neuronal death or dysfunction is varied and not fully understood but upon injury or upon ischemic insult, damaged neurons release massive amounts of the neurotransmitter glutamate, which is excitotoxic to the surrounding neurons (Choi et al., (1988), Neuron 1: 623-634; Rothman et al., (1984), J. Neurosci. 4: 1884-1891; Choi end Rothman, (1990), Ann. Rev. Neurosci. 13: 171-182; David et al., (1988), Exp. Eye Res. 46:657-662; Drejer et al., (1985), J. Neurosci. 45:145-151. Glutamate is a negatively charged amino acid that is an excitatory synaptic transmitter in the mammalian nervous system. Although the concentration of glutamate can reach the millimolar range in nerve terminals its extracellular concentration is maintained at a low level to prevent neurotoxicity. It has been noted that glutamate can be toxic to neurons if presented at a high concentration. The term “excitotoxicity” has been used to describe the cytotoxic effect that glutamate (and other such excitatory amino acids) can have on neurons when applied at high dosages.

Physiologically, excessive release, inhibition of uptake, or both can achieve high levels of glutamate. Normally, a low concentration of extracellular glutamate is maintained by both neurons and astrocytes. Neurons store glutamate in intracellular stores and regulate its release. See, Reagan, R. F., Excitotoxicity and Central Nervous System Trauma, in The Neurobiology of Central Nervous Trauma, New York, Oxford University Press, 1994, pp. 173-181 (Salzman S K, Faden A I, eds). Astrocytes take up extracellular glutamate by specific transporters and convert the glutamate into glutamine that is then released for neuronal uptake. See, Robinson, M. B. & Dowd L A, Adv Pharmacol, 1997; 37:69-115. In the process of excitotoxicity, glutamate is released in a self-perpetuating manner by the neurons, resulting in excessive or prolonged activation of glutamate receptors.

The conjunction of such excessive glutamate stimulation on the energy-depleted neurons taken with the compromised ability of the neurosupportive astrocytes to sequester toxic levels of extracellular glutamate leads to neuronal death via necrosis and apoptosis. Various interventions are currently being examined to reduce neuronal death associated with central nervous system injuries and diseases. In addition, in many chronic neurodegenerative conditions, inflammation and oxidative stress are key components of the pathology. See, Kermer et al., Cell Tissue Res 298:383-395, 1999. Such therapies include glutamate release inhibitors, glutamate receptor antagonists, Ca2+ channel blockers, GABA receptor agonists, gangliosides, neurotrophic factors, calpain inhibitors, caspase inhibitors, free radical scavengers, immuno- and cell metabolism modulators.

For example, several studies have shown the involvement of glutamate in the pathophysiology of: 1) Huntington's disease (HD) (Coyle and Schwartz, (1976), Nature 263: 244-246; 2) Alzheimer's disease (AD) (Maragos et al, (1987), TINS 10: 65-68; 3) Epilepsy (Nadler et al, (1978), Nature 271: 676-677); 4) Lathyrism (Spencer et al, (1986), Lancet 239: 1066-1067; 5) Amyotropic Lateral Sclerosis (ALS) and Parkinsonian dementia of Guam (Caine et al, (1986), Lancet 2: 1067-1070) as well as in the neuropathology associated with stroke, ischemia and reperfusion (See, Dykens et al, (1987), J. Neurochem. 49: 1222-1228).

Thus, injury to neurons may be caused by overstimulation of receptors by excitatory amino acids including glutamate and aspartate (See, Lipton et al. (1994) New Engl. J. Med. 330:613 621). Indeed, the N-methyl-D-aspartate (NMDA) subtype of glutamate receptor is suggested to have many important roles in normal brain function, including synaptic transmission, learning and memory, and neuronal development (See, Lipston et al. (1994) supra; Meldrum et al. (1990) Trends Pharm. Sci. 11:379-387). However, over-stimulation of the NMDA subtype of glutamate receptor leads to increased free radical production and neuronal cell death, which can be modulated by antioxidants (See, Herin et al. (2001) J. Neurochem. 78:1307-1314; Rossato et al. (2002) Neurosci. Lett. 318:137-140).

Thus the death or dysfunction of neurons and supporting cells in the cochlea or the vestibular system or the auditory or vestibular nerves including the primary auditory receptors, the hair cells in the Organ of Corti, and the primary auditory neurons and the spiral ganglion neurons in the cochlea, the neurons of the eight nerve and the vestibular ganglion neurons, can produce profound and long-lasting hearing loss and/or vestibular disturbances in mammals including humans. Thus there is a need for the development of methods and compounds that can protect the cells of the mammalian inner ear from this degeneration, i.e., are otoprotective.

### SUMMARY OF THE INVENTION

The present invention relates in general to neuroprotective methods, and more specifically to methods and compounds for prevention of damage to neural tissue and cells of the mammalian inner ear that are derived from the same neurogenic ectoderm, resulting from injury, trauma, toxins or acute or chronic disease processes.

This invention is based, in part, on the discovery that the administration of one or more members of a family of carbamate compounds provides a general neuroprotective effect on the mammalian nervous system and this protective effect would also include the components of the mammalian inner ear that are derived from same neurogenic ectoderm. This would include, but not be limited to, those neurons, supporting and related cells in the cochlea or the vestibular system or the auditory or vestibular nerves that during embryogenesis are derived from the same neurogenic ectoderm, the otic placode, including; the vestibular ganglion, the spiral ganglion neurons in the cochlea, the otic vesicle, the primary auditory receptors, i.e., the hair cells in the Organ of Corti, and the primary auditory neurons and the neurons of the eight nerve.

Neuroprotection provided by this invention includes protection from damage resulting from neural injury or insult and from neurotoxic or ototoxic agents. Thus, neuroprotection or otoprotection provided by this invention will be useful in the treatment of toxic damage from ototoxic drugs and acute and chronic degenerative disorders including including those affecting the cochlea and vestibular system including the hair cells and the eighth nerve

Otoprotection provided by this invention may be brought about upon injured or diseased tissue or in a preventative fashion during or prior to events expected to lead to a traumatic insult.

The invention provides methods for providing otoprotection; for inhibiting cell degeneration or cell death; for treatment or prophylaxis of a otodegenerative disease; or for ameliorating the otototoxic effect of a compound, for example, a toxin; or therapeutic compound that exerts a otototoxic side effect, e.g. antibiotics or chemotherapy agents, in a subject in need thereof, by administering to the subject an effective amount of a compound of the invention, or it's pharmaceutically acceptable salt or ester either alone or in combination with another medication along with a pharmaceutically acceptable excipient.

In various embodiments, the subject, for example, a human, may be suffering from insult or injury to the inner ear; or may be suffering from a condition selected from; ototoxic drug exposure, trauma, tinnitus, Meniere's Disease including immune-mediated Meniere's disease, immune-mediated cochlear or vestibular disorders (IMCVD), autoimmune ear disease (AIED), Cogan's Syndrome, and ischemic events affecting the inner ear or eight nerve.

Accordingly, the present invention provides methods for providing otoprotection comprising administering to a subject in need thereof a therapeutically effective amount of a composition that comprises at least one compound having Formula 1 or Formula 2:

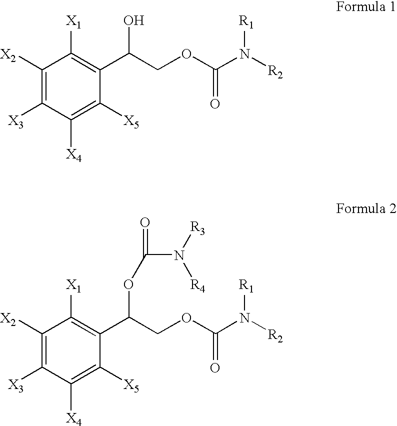

wherein R₁, R₂, R₃, and R₄ are, independently, hydrogen or C₁-C₄ alkyl; and X₁, X₂, X₃, X₄, and X₅ are, independently, hydrogen, fluorine, chlorine, bromine or iodine. The said C₁-C₄ alkyl group of Formula 1 or Formula 2 can be substituted or unsubstituted. In one aspect of the present invention, the C₁-C₄ alkyl group is substituted with a phenyl group. The phenyl group can be unsubstituted or substituted. In certain embodiments, the phenyl group is unsubstituted or substituted with halogen, C₁-C₄ alkyl, C₁-C₄ alkoxy, amino, nitro, or cyano.

In the present invention, X₁, X₂, X₃, X₄, and X₅ can be hydrogen, fluorine, chlorine, bromine or iodine. In certain embodiments, X₁, X₂, X₃, X₄, and X₅ are, independently, hydrogen or chlorine. In a preferred embodiment of the present invention, X₁ is fluorine, chlorine, bromine or iodine. In one aspect, X₁ is chlorine and X₂, X₃, X₄, and X₅ are, independently, hydrogen. In another preferred embodiment, R₁, R₂, R₃, and R₄ are, independently, hydrogen.

The present invention provides enantiomers of Formula 1 or Formula 2 for providing otoprotection in a subject. In certain embodiments, a compound of Formula 1 or Formula 2 will be in the form of a single enantiomer thereof. In other embodiments, a compound of Formula 1 or Formula 2 will be in the form of an enantiomeric mixture in which one enantiomer predominates with respect to another enantiomer. In one aspect, the enantiomer will predominate to the extent of 90% or greater or to the extent of 98% or greater.

The present invention also provides methods comprising administering to a subject a otoprotective amount of a composition that comprises at least one compound having Formula 1 or Formula 2 wherein R₁, R₂, R₃, and R₄ are, independently, hydrogen or C₁-C₄ alkyl; and X₁, X₂, X₃, X₄, and X₅ are, independently, hydrogen, fluorine, chlorine, bromine or iodine. In one embodiment, before administration of the composition to the subject, a determination will be made as to whether or not the subject suffers from some form of acute or chronic otodegeneration or inner ear injury from, e.g., toxic drugs.

The present invention also provides methods comprising identifying a patient at risk of developing acute or chronic otodegeneration or inner ear system injury or a patient in need of treatment with a otoprotective drug (OPD), as defined below and administering a composition that comprises at least one compound having Formula 1 or Formula 2 to the subject.

In certain embodiments of the present invention, a therapeutically effective amount of a compound having Formula 1 or Formula 2 for providing neuroprotection is from about 0.01 mg/Kg/dose to about 150 mg/Kg/dose.

In certain embodiments a therapeutically effective amount of pharmaceutical composition for providing otoprotection comprising one or more of the enantiomers of this invention or a pharmaceutically acceptable salt or ester thereof and a pharmaceutically acceptable carrier or excipient is administered to a subject or patient in need of treatment with a otoprotective drug or OPD.

Pharmaceutical compositions comprising at least one compound having Formula 1 or Formula 2 are administered to subjects in need thereof. In certain embodiments, a subject or patient in need of treatment with a otoprotective drug or OPD may be one who has experienced some form of acute trauma or injury to the neural derived cells of the inner ear or who has some form of acute or chronic otodegenerative disorder. In one aspect, the subject or patient will be determined to be at risk for developing an acute or chronic otodegenerative disorder at the time of administration, i.e., a patient in need of treatment with a otoprotective drug. In other embodiments, a subject in need thereof is one who has acute injury or trauma to the cells of their inner ear at the time of administration.

### BRIEF DESCRIPTION OF THE FIGURES

FIG. 1. Effects of increasing doses of test compound on the number of neurons in different areas of the hippocampus counted at 14 days after Li-Pilo SE. Values are expressed as the number of neuronal cell bodies in each area of interest.

o p<0.05, p<0.01, statistically significant difference between test compound and control rats; # p<0.05, * p<0.01, statistically significant differences between test compound and DZP rats.

FIG. 2. Effects of increasing doses of test compound on the number of neurons in different areas of the cerebral cortex counted at 14 days after Li-Pilo SE. Values are expressed as the number of neuronal cell bodies in each area of interest.

o p<0.05, p<0.01, statistically significant difference between test compound and control rats; # p<0.05, * p<0.01, statistically significant differences between test compound and DZP rats.

Abbreviations: D: dorsal, V: ventral.

FIG. 3. Variable neuroprotective effects of increasing doses of test compound on the number of neurons in the piriform cortex counted at 14 days after Li-Pilo SE. Values are expressed as the number of neuronal cell bodies in each area of interest.

o p<0.05, p<0.01, statistically significant difference between test compound and control rats; # p<0.05, * p<0.01, statistically significant differences between test compound and DZP rats.

e p<0.05, ∞ p<0.01, statistically significant differences between the two subgroups of rats (A and B) treated by a given dose of test compound.

Abbreviations: D: dorsal, V: ventral.

FIG. 4. Effects of increasing doses of test compound on the mean latency to the first SRS (A) and on the number of neurons remaining in layers III/IV of the entorhinal cortex (B). The number of animals in each group or subgroup is indicated over each bar.

There is a clear subdivision in the groups treated with 60, 90 and 120 mg/kg test compound with about half of the animals exhibiting a prolonged latency to epilepsy that seems to correlate with the number of surviving neurons in layers III/IV of the entorhinal cortex.

### DETAILED DESCRIPTION OF THE INVENTION

Patients with injury or damage of any kind to the cells of inner ear that are derived from neural tissue, including but not be limited to those neurons, supporting and related cells in the cochlea or the vestibular system or the auditory or vestibular nerves that during embryogenesis are derived from the same neurogenic ectoderm, the otic placode, including; the vestibular ganglion, the spiral ganglion neurons in the cochlea, the otic vesicle, the primary auditory receptors, the hair cells in the Organ of Corti, and the primary auditory neurons and the neurons of the eight nerve may benefit from these the neuroprotective method of this invention. This nervous system injury may take the form of an abrupt insult or an acute injury to the these cells as in, for example, acute otodegenerative disorders and ototoxic drugs, hypoxia-ischemia or the combination thereof resulting in cell death or compromise.

Injury includes, but is not limited to, insult or injury to the inner ear which may result from a condition selected from; ototoxic drug exposure, trauma, tinnitus, Meniere's Disease including immune-mediated Meniere's disease, immune-mediated cochlear or vestibular disorders (IMCVD), autoimmune ear disease (AIED), Cogan's Syndrome, and ischemic events affecting the inner ear or eight nerve, blunt or penetrating head trauma. In addition, deprivation of oxygen or blood supply in general can cause acute injury as in hypoxia and/or ischemia including, but is not limited to, ischemia or infarction of the auditory nerve or related structures.

In one preferred embodiment the compounds of the invention would be used to provide otoprotection in disorders involving trauma and progressive injury to the neuronal and neuronal derived cells of the inner ear resulting form the administration of ototoxic drugs including but not limited to antibiotics and chemotherapy agents.

Therefore, the term “otoprotection” or “otoprotective” as used herein shall mean; inhibiting, preventing, ameliorating or reducing the severity of the dysfunction, degeneration or death of: nerve cells; axons or their supporting or related cells, including cells derived during embryogenesis from the same neurogenic ectoderm, the otic placode including the neurons of the eight or auditory nerve, the cochlea; the vestibular system, the vestibular nerve; the primary auditory receptors, the hair cells in the Organ of Corti; the primary auditory neurons; the spiral ganglion neurons “SGN” in the cochlea and the vestibular ganglion neurons (“VGN”), of a mammal, including a human. This includes the treatment or prophylaxis of trauma, degenerative diseases and protection against the ototoxic effect of a drug or toxin (for example, antibiotics and chemotherapy agents or other toxins.

Therefore, the term “a patient in need of treatment with a otoprotective drug (OPD)” as used herein will refer to any patient who currently has or may develop any of the above syndromes or disorders or who requires or has had ototoxic drug administration, or any disorder in which the patient's present clinical condition or prognosis could benefit from providing otoprotection to prevent the; development, extension, worsening or increased resistance to treatment of any disease or disorder involving the neurons of the eight nerve; the cochlea; the vestibular system; the primary auditory receptors, the hair cells in the Organ of Corti; the primary auditory neurons; the spiral ganglion neurons “SGN” in the cochlea and the vestibular ganglion neurons (“VGN”) of a mammal, including a human.

The term “treating” or “treatment” as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination and/or neurological examination. Accordingly, the term “treating” or “treatment” includes the administration of the compounds or agents of the present invention to provide otoprotection

The term “therapeutic effect” as used herein, refers to the effective provision of otoprotection effects to prevent or minimize the death or damage or dysfunction of the cells of the patient's inner ear.

The term “a therapeutically effective amount” as used herein means a sufficient amount of one or more of the compounds of the invention to produce a therapeutic effect, as defined above, in a subject or patient in need of such otoprotection treatment.

The terms “subject” or “patient” are used herein interchangeably and as used herein mean any mammal including but not limited to human beings including a human patient or subject to which the compositions of the invention can be administered. The term mammals include human patients and non-human primates, as well as experimental animals such as rabbits, rats, and mice, and other animals.

In some embodiments the methods of the present invention will be advantageously used to treat a patient who is not suffering or known to be suffering from a condition that is known in the art to be effectively treated with carbamate compounds. In these cases the decision to use the methods and compounds of the present invention would be made on the basis of determining if the patient is a “patient in need of treatment with a otoprotective drug (OPD)”, as that term is defined above.

In some embodiments this invention provides methods of otoprotection. In certain embodiments, these methods comprise administering a therapeutically effective amount of a carbamate compound of the invention to a patient who has not yet developed overt, clinical signs or symptoms of injury or damage to the cells of the inner ear but who may be in a high risk group for the development of such damage because of disease, injury or trauma or because of the need to administer ototoxic drugs or because of some known predisposition either biochemical or genetic or the finding of a verified biomarker of one or more of these disorders.

Thus, in some embodiments, the methods and compositions of the present invention are directed toward otoprotection in a subject who is at risk of developing inner ear damage but has not developed clinical evidence. This patient may simply be at “greater risk” as determined by the recognition of any factor in a subject's, or their families, medical history, physical exam or testing that is indicative of a greater than average risk for developing inner ear damage. Therefore, this determination that a patient may be at a “greater risk” by any available means can be used to determine whether the patient should be treated with the methods of the present invention.

Accordingly, in an exemplary embodiments, subjects who may benefit from treatment by the methods and compounds of this invention can be identified using accepted screening methods to determine risk factors for inner ear damage. These screening methods include, for example, conventional work-ups to determine risk factors and treatment with medications that are ototoxic.

The determination of which patients may benefit from treatment with an OPD in patients who have no clinical signs or symptoms may be based on a variety of “surrogate markers” or “biomarkers”.

As used herein, the terms “surrogate marker” and “biomarker” are used interchangeably and refer to any anatomical, biochemical, structural, electrical, genetic or chemical indicator or marker that can be reliably correlated with the present existence or future development of neuronal or inner ear damage. In some instances, brain-imaging techniques, such as computer tomography (CT), magnetic resonance imaging (MRI) or positron emission tomography (PET), can be used to determine whether a subject is at risk for neuronal damage.

It is expected that many more such biomarkers utilizing a wide variety of detection techniques will be developed in the future. It is intended that any such marker or indicator of the existence or possible future development of neuronal damage, as the latter term is used herein, may be used in the methods of this invention for determining the need for treatment with the compounds and methods of this invention.

A determination that a subject has, or may be at risk for developing, inner ear damage would also include, for example, a medical evaluation that includes a thorough history, a physical examination, and a series of relevant bloods tests. It can also include an electroencephalogram (EEG), CT, MRI or PET scan.

### The Carbamate Compounds of the Invention

The present invention provides methods of using 2-phenyl-1,2-ethanediol monocarbomates and dicarbamates to provide otorotection to patients in need thereof.

Suitable methods for synthesizing and purifying carbamate compounds, including carbamate enantiomers, used in the methods of the present invention are well known to those skilled in the art. For example, pure enantiomeric forms and enantiomeric mixtures of 2-phenyl-1,2-ethanediol monocarbomates and dicarbamates are described in U.S. Pat. Nos. 5,854,283, 5,698,588, and 6,103,759, the disclosures of which are herein incorporated by reference in their entirety.

Representative carbamate compounds according to the present invention include those having Formula 1 or Formula 2:

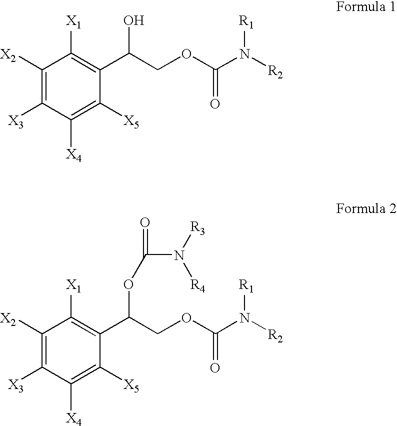

wherein R₁, R₂, R₃, and R₄ are, independently, hydrogen or C₁-C₄ alkyl and X₁, X₂, X₃, X₄, and X₅ are, independently, hydrogen, fluorine, chlorine, bromine or iodine.

“C₁-C₄ alkyl” as used herein refers to substituted or unsubstituted aliphatic hydrocarbons having from 1 to 4 carbon atoms. Specifically included within the definition of “alkyl” are those aliphatic hydrocarbons that are optionally substituted. In a preferred embodiment of the present invention, the C₁-C₄ alkyl is either unsubstituted or substituted with phenyl.

The term “phenyl”, as used herein, whether used alone or as part of another group, is defined as a substituted or unsubstituted aromatic hydrocarbon ring group having 6 carbon atoms. Specifically included within the definition of “phenyl” are those phenyl groups that are optionally substituted. For example, in a preferred embodiment of the present invention, the, “phenyl” group is either unsubstituted or substituted with halogen, C₁-C₄ alkyl, C₁-C₄ alkoxy, amino, nitro, or cyano.

In a preferred embodiment of the present invention, X₁ is fluorine, chlorine, bromine or iodine and X₂, X₃, X₄, and X₅ are hydrogen.

In another preferred embodiment of the present invention, X₁, X₂, X₃, X₄, and X₅ are, independently, chlorine or hydrogen.

In another preferred embodiment of the present invention, R₁, R₂, R₃, and R₄ are all hydrogen.

It is understood that substituents and substitution patterns on the compounds of the present invention can be selected by one of ordinary skill in the art to provide compounds that are chemically stable and that can be readily synthesized by techniques known in the art as well as the methods provided herein.

Representative 2-phenyl-1,2-ethanediol monocarbomates and dicarbamates include, for example, the following compounds:

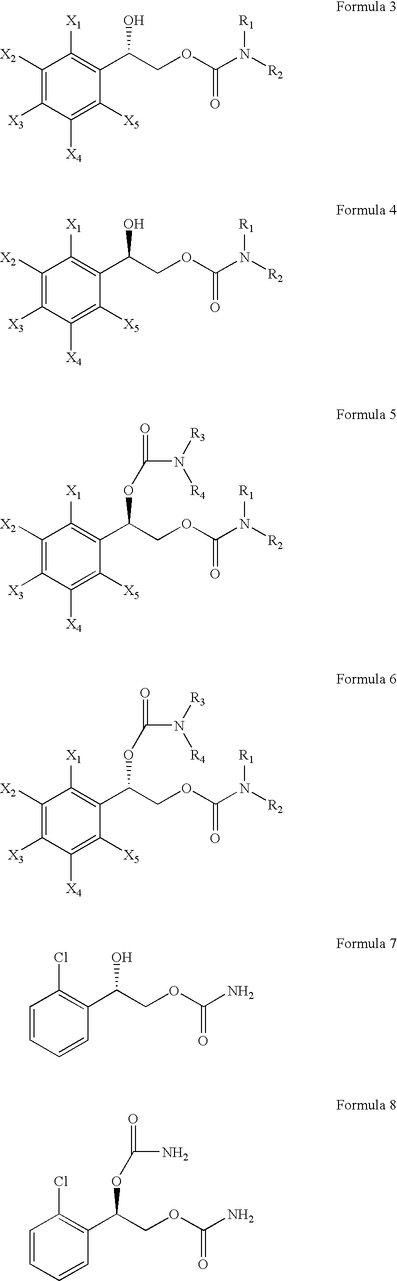

The present invention includes the use of isolated enantiomers of Formula 1 or Formula 2. In one preferred embodiment, a pharmaceutical composition comprising the isolated S-enantiomer of Formula 1 is used to provide neuroprotection in a subject. In another preferred embodiment, a pharmaceutical composition comprising the isolated R-enantiomer of Formula 2 is used to provide neuroprotection in a subject. In another embodiment, a pharmaceutical composition comprising the isolated S-enantiomer of Formula 1 and the isolated R-enantiomer of Formula 2 can be used to provide neuroprotection in a subject.

The present invention also includes the use of mixtures of enantiomers of Formula 1 or Formula 2. In one aspect of the present invention, one enantiomer will predominate. An enantiomer that predominates in the mixture is one that is present in the mixture in an amount greater than any of the other enantiomers present in the mixture, e.g., in an amount greater than 50%. In one aspect, one enantiomer will predominate to the extent of 90% or to the extent of 91%, 92%, 93%, 94%, 95%, 96%, 97% or 98% or greater. In one preferred embodiment, the enantiomer that predominates in a composition comprising a compound of Formula 1 is the S-enantiomer of Formula 1. In another preferred embodiment, the enantiomer that predominates in a composition comprising a compound of Formula 2 is the R-enantiomer of Formula 2.

In a preferred embodiment of the present invention, the enantiomer that is present as the sole enantiomer or as the predominate enantiomer in a composition of the present invention is represented by Formula 3 or Formula 5, wherein X₁, X₂, X₃, X₄, X₅, R₁, R₂, R₃, and R₄ are defined as above, or by Formula 7 or Formula 8.

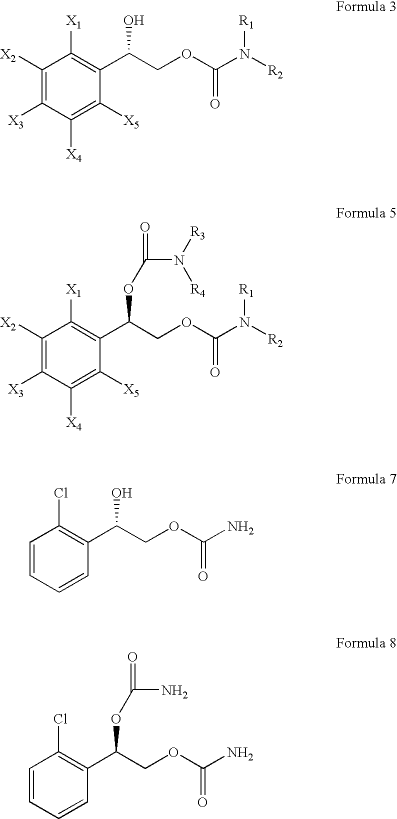

The present invention provides methods of using enantiomers and enantiomeric mixtures of compounds represented by Formula 1 and Formula 2. A carbamate enantiomer of Formula 1 or Formula 2 contains an asymmetric chiral carbon at the benzylic position, which is the aliphatic carbon adjacent to the phenyl ring.

An enantiomer that is isolated is one that is substantially free of the corresponding enantiomer. Thus, an isolated enantiomer refers to a compound that is separated via separation techniques or prepared free of the corresponding enantiomer. The term “substantially free,” as used herein, means that the compound is made up of a significantly greater proportion of one enantiomer. In preferred embodiments, the compound includes at least about 90% by weight of a preferred enantiomer. In other embodiments of the invention, the compound includes at least about 99% by weight of a preferred enantiomer. Preferred enantiomers can be isolated from racemic mixtures by any method known to those skilled in the art, including high performance liquid chromatography (HPLC) and the formation and crystallization of chiral salts, or preferred enantiomers can be prepared by methods described herein.

Methods for the preparation of preferred enantiomers would be known to one of skill in the art and are described, for example, in Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972).

Additionally, compounds of the present invention can be prepared as described in U.S. Pat. No. 3,265,728 (the disclosure of which is herein incorporated by reference in its entirety and for all purposes), U.S. Pat. No. 3,313,692 (the disclosure of which is herein incorporated by reference in its entirety and for all purposes), and the previously referenced U.S. Pat. Nos. 5,854,283, 5,698,588, and 6,103,759 (the disclosures of which are herein incorporated by reference in their entirety and for all purposes).

### Carbamate Compounds as Pharmaceuticals:

The present invention provides enantiomeric mixtures and isolated enantiomers of Formula 1 and/or Formula 2 as pharmaceuticals. The carbamate compounds are formulated as pharmaceuticals to provide neuroprotection in a subject.

In general, the carbamate compounds of the present invention can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection.) Administration of the compounds directly to the nervous system can include, for example, administration to intracerebral, intraventricular, intacerebroventricular, intrathecal, intracisternal, intraspinal or peri-spinal routes of administration by delivery via intracranial or intravertebral needles or catheters with or without pump devices.

Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso A R: Remington's Pharmaceutical Sciences, 17th ed., Mack Publishing Company, Easton Pa., 1985, the disclosure of which is incorporated herein by reference in its entirety and for all purposes. Suitable liquid carriers, especially for injectable solutions, include water, aqueous saline solution, aqueous dextrose solution, and glycols.

The carbamate compounds can be provided as aqueous suspensions. Aqueous suspensions of the invention can contain a carbamate compound in admixture with excipients suitable for the manufacture of aqueous suspensions. Such excipients can include, for example, a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate).

The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions for use in the present methods can be formulated by suspending a carbamate compound in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these.

Suitable emulsifying agents include naturally occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations of the present invention suitable for parenteral administration, such as, for example, by intraarticular (in the joints), intravenous, intramuscular, intradermal, intraperitoneal, and subcutaneous routes, can include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter.

Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The concentration of a carbamate compound in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally acceptable diluents or solvent, such as a solution of 1,3-butanediol. The formulations of commends can be presented in unit-dose or multi-dose sealed containers, such as ampoules and vials. Injection solutions and suspensions can be prepared from sterile powders, granules, and tablets of the kind previously described.

A carbamate compound suitable for use in the practice of this invention can be and is preferably administered orally. The amount of a compound of the present invention in the composition can vary widely depending on the type of composition, size of a unit dosage, kind of excipients, and other factors well known to those of ordinary skill in the art. In general, the final composition can comprise, for example, from 1.0 percent by weight (% w) to 95% w of the carbamate compound, preferably 10.0% w to 90% w, with the remainder being the excipient or excipients.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient.

Formulations suitable for oral administration can consist of (a) liquid solutions, such as an effective amount of the pharmaceutical formulation suspended in a diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions.

Pharmaceutical preparations for oral use can be obtained through combination of the compounds of the present invention with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxymethyl cellulose, hydroxypropylmethyl-cellulose or sodium carboxymethylcellulose; and gums including arabic and tragacanth; as well as proteins such as gelatin and collagen. If desired, disintegrating or solubilizing agents can be added, such as the cross-linked polyvinyl pyrrolidone, agar, alginic acid, or a salt thereof, such as sodium alginate. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compounds of the present invention can also be administered in the form of suppositories for rectal administration of the drug. These formulations can be prepared by mixing the drug with a suitable non-irritating excipient that is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The compounds of the present invention can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug (e.g., mifepristone)-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention.

In another embodiment, the compounds of the present invention can be delivered by the use of liposomes which fuse with the cellular membrane or are endocytosed, i.e., by employing ligands attached to the liposome that bind to surface membrane protein receptors of the cell resulting in endocytosis. By using liposomes, particularly where the liposome surface carries ligands specific for target cells, or are otherwise preferentially directed to a specific organ, one can focus the delivery of the carbamate compound into target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation can be a lyophilized powder which can contain, for example, any or all of the following: 1 mM-50 mM histidine, 0.1%-2% sucrose, 2%-7% mannitol, at a pH range of 4.5 to 5.5, that is combined with buffer prior to use.

Pharmaceutically acceptable salts and esters refers to salts and esters that are pharmaceutically acceptable and have the desired pharmacological properties. Such salts include salts that may be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N methylglucamine, and the like.

Pharmaceutically acceptable salts can also include acid addition salts formed from the reaction of amine moieties in the parent compound with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds. When there are two acidic groups present, a pharmaceutically acceptable salt or ester may be a mono-acid-mono-salt or ester or a di-salt or ester; and similarly where there are more than two acidic groups present, some or all of such groups can be salified or esterified.

Compounds named in this invention can be present in unsalified or unesterified form, or in salified and/or esterified form, and the naming of such compounds is intended to include both the original (unsalified and unesterified) compound and its pharmaceutically acceptable salts and esters. The present invention includes pharmaceutically acceptable salt and ester forms of Formula 1 and Formula 2. More than one crystal form of an enantiomer of Formula 1 or Formula 2 can exist and as such are also included in the present invention.

A pharmaceutical composition of the invention can optionally contain, in addition to a carbamate compound, at least one other therapeutic agent useful in the treatment of a disease or condition associated with providing neuroprotection.

Methods of formulating pharmaceutical compositions have been described in numerous publications such as Pharmaceutical Dosage Forms: Tablets. Second Edition. Revised and Expanded. Volumes 1-3, edited by Lieberman et al; Pharmaceutical Dosage Forms: Parenteral Medications. Volumes 1-2, edited by Avis et al; and Pharmaceutical Dosage Forms: Disperse Systems. Volumes 1-2, edited by Lieberman et al; published by Marcel Dekker, Inc, the disclosure of which are herein incorporated by reference in their entireties and for all purposes.

The pharmaceutical compositions are generally formulated as sterile, substantially isotonic and in full compliance with all Good Manufacturing Practice (GMP) regulations of the U.S. Food and Drug Administration.

### Dosage Regimens

The present invention provides methods of providing neuroprotection in a mammal using carbamate compounds. The amount of the carbamate compound necessary to provide neuroprotection is defined as a therapeutically or a pharmaceutically effective dose. The dosage schedule and amounts effective for this use, i.e., the dosing or dosage regimen will depend on a variety of factors including the stage of the disease, the patient's physical status, age and the like. In calculating the dosage regimen for a patient, the mode of administration is also taken into account.

A person of ordinary skill in the art will be able without undue experimentation, having regard to that skill and this disclosure, to determine a therapeutically effective amount of a particular substituted carbamate compound for practice of this invention (see, e.g., Lieberman, Pharmaceutical Dosage Forms (Vols. 1-3, 1992); Lloyd, 1999, The art, Science and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds.

For treatment purposes, the compositions or compounds disclosed herein can be administered to the subject in a single bolus delivery, via continuous delivery over an extended time period, or in a repeated administration protocol (e.g., by an hourly, daily or weekly, repeated administration protocol). The pharmaceutical formulations of the present invention can be administered, for example, one or more times daily, 3 times per week, or weekly. In one embodiment of the present invention, the pharmaceutical formulations of the present invention are orally administered once or twice daily.

A treatment regimen with the compounds of the present invention can commence, for example, after a subject suffers from a damaging injury or other initial insult but before the subject is diagnosed with an inner ear disorder or other manifestation of injury. In one embodiment, a subject that is identified as being at a high risk of developing inner ear injury or a subject having a disease associated with a risk of developing inner ear damage, e.g., tinnitus, Meniers' Disease or exposure to ototoxic drugs, can commence a treatment regimen with a carbamate compound of the present invention.

In this context, a therapeutically effective dosage of the biologically active agent(s) can include repeated doses within a prolonged treatment regimen that will yield clinically significant results to provide neuroprotection. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

In an exemplary embodiment of the present invention, unit dosage forms of the compounds are prepared for standard administration regimens. In this way, the composition can be subdivided readily into smaller doses at the physician's direction. For example, unit dosages can be made up in packeted powders, vials or ampoules and preferably in capsule or tablet form.

The active compound present in these unit dosage forms of the composition can be present in an amount of, for example, from about 10 mg. to about one gram or more, for single or multiple daily administration, according to the particular need of the patient. By initiating the treatment regimen with a minimal daily dose of about one gram, the blood levels of the carbamate compounds can be used to determine whether a larger or smaller dose is indicated.

Effective administration of the carbamate compounds of this invention can be administered, for example, at an oral or parenteral dose of from about 0.1 mg/kg/dose to about 50 mg/kg/dose. Preferably, administration will be from about 1.0/mg/kg/dose to about 25 mg/kg/dose, more preferably from about 2.0 to about 40 mg/kg/dose. Therefore, the therapeutically effective amount of the active ingredient contained per dosage unit as described herein can be, for example, from about 140 mg/day to about 2800 mg/day for a subject having, for example, an average weight of 70 kg.

The methods of this invention also provide for kits for use in providing neuroprotection. After a pharmaceutical composition comprising one or more carbamate compounds of this invention, with the possible addition of one or more other compounds of therapeutic benefit, has been formulated in a suitable carrier, it can be placed in an appropriate container and labeled for providing neuroprotection. Additionally, another pharmaceutical comprising at least one other therapeutic agent useful in the provide neuroprotection, treatment of epileptogenesis, epilepsy or another disorder or condition associated with neuronal injury can be placed in the container as well and labeled for treatment of the indicated disease. Such labeling can include, for example, instructions concerning the amount, frequency and method of administration of each pharmaceutical.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. The following examples are provided to illustrate specific aspects of the invention and are not meant to be limitations.

### EXAMPLES

The following Examples are set forth to aid in the understanding of the invention, and are not intended and should not be construed to limit in any way the invention set forth in the claims which follow thereafter. The activities of a compound of Formula (I) and Formula (II) for use in providing neuroprotection were evaluated in the following experimental examples. In all the examples below, i.e., Examples 1, 2, 3 and 4 the activity of an isolated S-enantiomer of Formula 1 (this compound is shown as Formula 7 above and will be referred to in the examples below as test compound (or TC or tc), was evaluated in a rat model of induced epilepsy to determine the efficacy of the compound for neuroprotection and in the treatment of epileptogenesis in the model of temporal lobe epilepsy induced by lithium and pilocarpine in the rat (Examples 1 and 2). In addition, Examples 3 and 4 show the efficacy of the same test compound to provide neuroprotection in models of serum withdrawal and in the transient cerebral ischemia model in the rat. These examples show the ability of test compound to protect neuronal tissue from a wide variety of damaging events. These examples are intended to be a way of illustrating various embodiments of the invention but not intended to limit the invention in any way.

The structure of test compound is shown below;

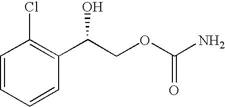

### Example 1

### The Lithium-Pilocarpine Model of Temporal Lobe Epilepsy

The model induced in rats by pilocarpine associated with lithium (Li-Pilo) reproduces most of the clinical and neurophysiological features of human temporal lobe epilepsy (Turski et al., 1989, Synapse 3:154-171; Cavalheiro, 1995, Ital J Neurol Sci 16:33-37). In adult rats, the systemic administration of pilocarpine leads to status epilepticus (SE). The lethality rate reaches 30-50% during the first days. In the surviving animals, neuronal damage predominates within the hippocampal formation, the piriform and entorhinal cortices, thalamus, amygdaloid complex, neocortex and substantia nigra. This acute seizure period is followed by a “silent” seizure-free phase lasting for a mean duration of 14-25 days after which all animals exhibit spontaneous recurrent convulsive seizures at the usual frequency of 2 to 5 per week (Turski et al., 1989, Synapse 3:154-171; Cavalheiro, 1995, Ital J Neurol Sci 16:33-37; Dube et al., 2001, Exp Neurol 167:227-241).

### Lithium-Pilocarpine and Treatments with the Test Compound

Male Wistar rats weighing 225-250 g, provided by Janvier Breeding Center (Le Genest-St-lste, France) were housed under controlled standard conditions (light/dark cycle, 7.00 a.m.-7.00 p.m. lights on), with food and water available ad libitum. All animal experimentation was performed in accordance with the rules of the European Communities Council Directive of Nov. 24, 1986 (86/609/EEC), and the French Department of Agriculture (License No 67-97). For electrode implantation, rats were anesthetized by an i.p. injection of 2.5 mg/kg diazepam (DZP, Valium, Roche, France) and 1 mg/kg ketamine chlorhydrate (Imalgene 1000, Rhone Merrieux, France). Four single-contact recording electrodes were placed on the skull, over the parietal cortex, two on each side.

### Status Epilepticus Induction:

### Treatment with the Test Compound and Occurrence of Spontaneous Recurrent Seizures (SRS)

All rats received lithium chloride (3 meq/kg, i.p., Sigma, St Louis, Mo., U.S.A.); about 20 h later, animals were placed into plexiglas boxes, in order to record baseline cortical EEG. Methylscopolamine bromide (1 mg/kg, s.c., Sigma) was administered to limit the peripheral effects of the convulsant. SE was induced by injecting pilocarpine hydrochloride (25 mg/kg, s.c., Sigma) 30 min after methyl-scopolamine. The bilateral EEG cortical activity was recorded during the whole duration of SE and behavioral changes were noted.

The effects of increasing doses of the test compound were studied on 3 groups of rats. The animals of the first group received 10 mg/kg of the test compound, i.p., 1 h after the onset of SE (pilo-test10) while the animals of groups 2 and 3 received 30 and 60 mg/kg of the test compound (pilo-test30 and pilo-test60), respectively.

Another group was injected with 2 mg/kg diazepam (DZP, i.m.) at 1 h after the onset of SE which are our standard treatment to improve animals survival after SE (pilo-DZP). The control group received saline instead of pilocarpine and the test compound (saline-saline). The pilo-test compound rats surviving SE were then injected about 10 h after the first test compound injection with a second i.p. injection of the same dose of the test compound and were maintained under a twice daily treatment with the test compound for 6 additional days. Pilo-DZP received a second injection of 1 mg/kg DZP on the day of SE at about 10 h after the first one. Thereafter, Pilo-DZP and saline-saline rats received twice daily an equivalent volume of saline.

The effects of the test compound on the EEG and on the latency to occurrence of SRS were investigated by daily video recording of the animals for 10 h per day and the recording of the electrographic activity twice a week for 8 h.

### Quantification of Cell Densities

Quantification of cell densities was performed at 6 days after SE on 8 pilo-DZP, 8 pilo-test10, 7 pilo-test30, 7 pilo-test60, and 6 saline-saline rats. At 14 days after SE, animals were deeply anesthetized with 1.8 g/kg pentobarbital (Dolethal®, Vetoquinol, Lure, France. Brains were then removed and frozen. Serial 20 μm slices were cut in a cryostat, air-dried during several days before thionine staining.

Quantification of cell densities was performed with a 10×10 boxes 1 cm² microscopic grid on coronal sections according to the stereotaxic coordinates of the rat brain atlas (Paxinos and Watson, 1986, The Rat brain in Stereotaxic Coordinates, 2^{nd} ed. Academic Press, San Diego). Cell counts were performed twice in a blind manner and were the average of at least 3 values from 2 adjacent sections in each individual animal. Counts involved only cells larger than 10 μm, smaller ones being considered as glial cells.

### Timm Staining

At 2 months after the onset of spontaneous recurrent seizures, mossy fiber sprouting was examined on rats in the chronic period exposed to the test compound or DZP and in 3 saline-saline rats. Animals were deeply anaesthetized and perfused transcardially with saline followed by 100 ml of 1.15% (w/v) Na₂S in 0.1 M phosphate buffer, and 100 ml of 4% (v/v) formaldehyde in 0.1 M phosphate buffer. Brains were removed from skull, post-fixed in 4% formaldehyde during 3-5 h and 40 μm sections were cut on a sliding vibratome and mounted on gelatin-coated slides.

The following day, sections were developed in the dark in a 26° C. solution of 50% (w/v) arabic gum (160 ml), sodium citrate buffer (30 ml), 5.7% (w/v) hydroquinone (80 ml) and 10% (w/v) silver nitrate (2.5 ml) during 40-45 min. The sections were then rinsed with tap water at 40° C. during at least 45 min, rinsed rapidly with distilled water and allowed to dry. They were dehydrated in ethanol and coverslipped.

Mossy fiber sprouting was evaluated according to criteria previously described in dorsal hippocampus (Cavazos et al., 1991, J Neurosci 11:2795-2803), which are follows: 0—no granules between the tips and crest of the DG; 1—sparse granules in the supragranular region in a patchy distribution between the tips and crest of DG; 2—more numerous granules in a continuous distribution between the tips and crest of DG; 3—prominent granules in a continuous pattern between tips and crest, with occasional patches of confluent granules between tips and crest; 4—prominent granules that form a confluent dense laminar band between tips and crest and 5—confluent dense laminar band of granules that extends into the inner molecular layer.

### Data Analysis

For the comparison of the characteristics of SE in pilo-saline and pilo-test compound animals, a non-paired Student's t-test was used. The comparison between the number of rats seizing in both groups was performed by means of a Chi square test. For neuronal damage, statistical analysis between groups was performed using ANOVA followed by a Fisher's test for multiple comparisons using the Statview software (Fisher R A, 1946a, Statistical Methods for Research Workers (10th edition) Oliver & Boyd, Edinburgh; Fisher R A, 1946b, The Design of Experiments (4th edition) Oliver & Boyd, Edinburgh) Behavioral and EEG characteristics of lithium-pitocarpine status epilepticus

A total number of Sprague-Dawley rats weighing 250-330 g were subjected to Li-pilo induced SE. The behavioral characteristics of SE were identical in both pilo-saline and pilo-test compound groups. Within 5 min after pilocarpine injection, rats developed diarrhea, piloerection and other signs of cholinergic stimulation. During the following 15-20 min, rats exhibited head bobbing, scratching, chewing and exploratory behavior. Recurrent seizures started around 15-20 min after pilocarpine administration. These seizures which associated episodes of head and bilateral forelimb myoclonus with rearing and falling progressed to SE at about 35-40 min after pilocarpine, as previously described (Turski et al., 1983, Behav Brain Res 9:315-335).

### EEG Patterns Suring SE

During the first hour of SE, in the absence of pharmacological treatment, the amplitude of the EEG progressively increased while the frequency decreased. Within 5 min after the injection of pilocarpine, the normal background EEG activity was replaced with low voltage fast activity in the cortex while theta rhythm (5-7 Hz) appeared in the hippocampus. By 15-20 min, high voltage fast activity superposed over the hippocampal theta rhythm and isolated high voltage spikes were recorded only in the hippocampus while the activity of the cortex did not substantially change.

By 35-40 min after pilocarpine injection, animals developed typical electrographic seizures with high voltage fast activity present in both the hippocampus and cortex which first occurred as bursts of activity preceding seizures and were followed by continuous trains of high voltage spikes and polyspikes lasting until the administration of DZP or the test compound. At about 3-4 h of SE, the hippocampal EEG was characterized by periodic electrographic discharges (PEDs, about one/sec) in the pilo-DZP and in the pilo-10 group in both hippocampus and cortex. The amplitude of EEG background activity was low in the pilo-test60 animals. By 6-7 h of SE, spiking activity was still present in the cortex and the hippocampus in the DZP- and test10-treated rats while the amplitude of the EEG decreased and came back to baseline levels in the hippocampus of test30 rats and in both structures of test60 treated rats. There was no difference between test10, test30, and test60 groups. By 9 h of SE, isolated spikes were still recorded in the hippocampus of test compound-treated rats and occasionally in the cortex. In both structures, the background activity was of very low amplitude at that time.

### Mortality Induced by SE

During the first 48 h after SE, the degree of mortality was similar in pilo-DZP rats (23%, 5/22), pilo-test10 rats (26%, 6/23), and pilo-test30 rats (20%, 5/25), The mortality rate was largely reduced in pilo-test60 rats in which it only reached 4% (1/23). The difference was statistically significant (p<0.01).

### EEG Characteristics of the Silent Phase and Occurrence of Spontaneous Recurrent Seizures

The EEG patterns during the silent period were similar in pilo-DZP and pilo-test10, 30 or 60 rats. At 24 and 48 h days after SE, the baseline EEG was still characterized by the occurrence of PEDs on which large waves or spikes could be superimposed. Between 1 and 8 h after injection of the test compound or vehicle injection, there was no change in the pilo-DZP or pilo-test10 groups. In test30 and test60 rats, the frequency and amplitude of PEDs decreased as soon as 10 min after injection and were replaced by spikes of large amplitude in the test30 group and of low amplitude in the test60 group. By 4 h after injection the EEG had returned to baseline levels in the two latter groups. By 6 days after SE, the EEG was still of lower amplitude than before pilocarpine injection and in most groups spikes could still be recorded, occasionally in the pilo-DZP, -test10 and -test30 rats. In pilo-test60 rats, the frequency of large amplitude spikes was higher than in all other groups. After the test compound or vehicle-injection, EEG recording was not affected by the injection in the pilo-DZP and pilo-test10 groups. In pilo-test30 rats, the injection induced the occurred of slow waves on the EEG of both hippocampus and cortex and a decreased frequency of spikes in the pilo-test60 rats.

All the rats exposed to DZP, test10 and test30 and studied until the chronic phase developed SRS with a similar latency. The latency was 18.2±6.9 days (n=9) in pilo-DZP rats, 15.4±5.1 days (n=7) in pilo-test10 rats, 18.9±9.0 days (n=10) in pilo-test30 rats. In the group of rats subjected to test60, a subgroup of rats became epileptic with a latency similar to that of the other groups, i.e. 17.6±8.7 days (n=7) while another group of rats became epileptic with a much longer delay ranging from 109 to 191 days post-SE (149.8±36.0 days, n=4) and one rat did not become epileptic in a delay of 9 months post-SE. The difference in the latency to SRS between pilo-DZP, pilo-test10, pilo-test30 and the first subgroup of pilo-TPM60 rats was not statistically significant. None of the saline-saline rats (n=5) developed SRS.

To calculate the frequency of SRS in pilocarpine-exposed rats, the seizure severity and distinguished stage III (clonic seizures of facial muscles and anterior limbs) and stage IV-V seizures (rearing and falling) was considered. The frequency of stage III SRS per week in pilo-DZP and pilo-test compound rats was variable amongst the groups. It was low, constant in the pilo-DZP and pilo-test60 (with early SRS onset) groups during the first 3 weeks and had disappeared during the 4th week in the pilo-DZP group. The frequency of stage III SRS was higher in the pilo-test10 group where it was significantly increased over pilo-DZP values during weeks 3 and 4. The frequency of more severe stage IV-V SRS was highest during the first week in most groups, except pilo-test30 and test60 with late seizure onset where the SRS frequency was constant over the whole 4 weeks in test30 group and over the first two weeks in the pilo-test60 group with late SRS onset in which no stage IV-V seizures where no seizures recorded after the second week. The frequency of stage IV-V SRS was significantly reduced in the test10, test30 and test60 (with early SRS onset) groups (2.3-6.1 SRS per week) compared to the pilo-DZP group (11.3 SRS per week) during the first week. During weeks 2-4-v the frequency of stage IV-V SRS was reduced in all groups compared to the first week reaching values of 2-6 seizures per week, except in the pilo-test60 group with early SRS onset where the frequency of seizures was significantly reduced to 0.6-0.9 seizure per week compared to the pilo-DZP group in which the frequency of SRS ranged from 3.3 to 5.8.

### Cell Densities in Hippocampus, Thalamus and Cortex

In pilo-DZP rats compared to saline-saline rats, the number of cells was massively decreased in the CA1 region of the hippocampus (70% cell loss in the pyramidal cell layer) while the CAS region was less extensively damaged (54% cell loss in CA3a and 31% in CA3b). In the dentate gyrus, the pilo-DZP rats experienced extensive cell loss in the hilus (73%) while the granule cell layer did not show visible damage. Similar damage was observed in the ventral hippocampus but cell counts were not performed in this region. Extensive damage was also recorded in the lateral thalamic nucleus (91% cell loss) while the mediodorsal thalamic nucleus was more moderately damaged (56%). In the piriform cortex, cell loss was total in layers III-IV which was no longer visible and reached 53% in layer II in pilo-DZP rats. In the dorsal entorhinal cortex, layers II and III-IV underwent slight damage (9 and 15%, respectively). Layer II of the ventral entorhinal cortex was totally preserved while layers III-IV underwent a 44% cell loss.

In the hippocampus of pilo-test compound animals, cell loss was reduced compared to pilo-DZP rats in the CA1 pyramidal layer in which the cell loss reached 75% in pilo-DZP and 35 and 16% in the pilo-test30 or pilo-test60 animals, respectively. This difference was statistically significant at the two test compound doses. In the CAS pyramidal layer, the test compound did not afford any protection in the CA3a area while the 60 mg/kg of the test compound dose was significantly neuroprotective in CA3b. In the dentate gyrus, the cell loss in the hilus was similar in pilo-test compound (69-72%) and pilo-DZP animals (73%). In the two thalamic nuclei, the 60 mg/kg dose was also protective in reducing neuronal damage by 65 and 42% in the lateral and mediodorsal nucleus, respectively. In the cerebral cortex, the treatment with the test compound afforded neuronal protection compared to DZP only at the highest dose, 60 mg/kg. At the two lowest doses, 10 and 30 mg/kg, the total loss of cells and tissue disorganization observed in layers III-IV of the piriform cortex was identical in pilo-DZP rats and pilo-test compound rats and did not allow any counting in any of the groups. In layers II and III-IV of the piriform cortex, the test60 treatment reduced neuronal damage recorded in the pilo-DZP rats by 41 and 44%, respectively. In the ventral entorhinal cortex, neuroprotection was induced by test60 administration in layers III-IV and reached 31% compared to pilo-DZP rats. In the entorhinal cortex, there was a slight worsening of cell loss in pilo-test10 rats compared with pilo-DZP rats in layers III-IV of the dorsal entorhinal cortex (28% more damage) and layers III-IV of the ventral entohinal cortex (35% more damage). At the other doses of the test compound, cell loss in the entorhinal cortex was similar to the one recorded in pilo-DZP rats.

### Mossy Fiber Sprouting in Hippocampus

All rats exhibiting SRS in pilo-DZP and pilo-TPM groups showed similar intensity of Timm staining in the inner molecular layer of the dentate gyrus (scores 2-4). Timm staining was present both on the upper and lower blades of the dentate gyrus. The mean value of the Timm score in the upper blade reached 2.8±0.8 in pilo-DZP rats (n=9), 1.5±0.6 in pilo-test10 rats (n=7), 2.6±1.0 in pilo-test30 rats (n=10), and 1.5±0.7 in the whole group of pilo-test60 rats (n=11). When the pilo-test60 group was subdivided according to the latency to SRS, the subgroup with early SRS occurrence showed a Timm score of 1.8±0.6 (n=6) and the subgroup of rats with late occurrence or absence of SRS had a Timm score of 1.2±0.6 (n=5). The values recorded in the pilo-DZP rats were statistically significantly different from the values in the pilo-test10 (p=0.032) and the pilo-test60 subgroup with late or no seizures (p=0.016).

### Discussion and Conclusions

The results of the present study show that a 7-day treatment with the test compound starting at 1 h after the onset of SE is able to protect some brain areas from neuronal damage, e.g., in the pyramidal cell layer of the CA1 and CA3b area, the mediodorsal thalamus, layers II and I1MV of the piriform cortex and layers III-IV of the ventral entorhinal cortex, but only at the highest dose the test compound, i.e. 60 mg/kg. The latter dose of the test compound is also able to delay the occurrence of SRS, at least in a subgroup of animals that became epileptic with a mean delay that was about 9-fold longer than in the other groups of animals and one animal did not become epileptic in a delay of 9 months after SE.

These results show that one compound with anti-ictal properties, which are the classical properties of most antiepileptic-marketed drugs, is also able to delay epileptogenesis, i.e. to be antiepileptogenic. The data of the present study show also that the test compound treatment, whatever the dose used, decreases the severity of the epilepsy since it decreases the number of stage IV-V seizures, mainly during the first week of occurrence and during the whole period of 4-weeks observation with the test60 treatment. Moreover, in the test10 group, there is a shift to an increase in the occurrence of less severe stage III seizures that are more numerous than in the pilo-DZP group.

### Example 2

### Potential Neuroprotective and Anti-Epileptogenic Effects of Increasing Doses of Test Compound in the Lithium-Pilocarpine Model of Epilepsy in Rats

### Introduction

The aim of the present project was to pursue our study on the potential neuroprotective and antiepileptogenic properties of test compound in the lithium-pilocarpine (Li-Pilo) model of temporal lobe epilepsy. This study follows a first one (Example 1 above) in which we showed that test compound was able to protect areas CA1 and CA3 of the hippocampus, piriform and ventral entorhinal cortex from neuronal damage induced by Li-Pilo status epilepticus (SE). Most of these neuroprotective properties occurred at the highest dose studied, 60 mg/kg and the treatment was able to delay the occurrence of spontaneous seizures in 36% (4 out of 11) of the rats. In the present study, we propose to study the consequences of treatment by test compound on neuronal damage, epileptogenesis, neuronal excitability and behavior.

### The Lithium-Pilocarpine Model of Temporal Lobe Epilepsy

The model induced in rats by pilocarpine associated with lithium (Li-Pilo) reproduces most of the clinical and neurophysiological features of human temporal lobe epilepsy (See, Turski et al., 1989; Synapse 3:154-171; Cavalheiro, 1995, Ital J Neurol Sci 16:33-37). In adult rats, the systemic administration of pilocarpine leads to SE that may last for up to 24 h. The lethality rate reaches 30-50% during the first days. In the surviving animals, neuronal damage predominates within the hippocampal formation, the piriform and entorhinal cortices, thalamus, amygdaloid complex, neocortex and substantia nigra. This acute seizure period is followed by a “silent” seizure-free phase lasting for a mean duration of 14-25 days after which all animals exhibit spontaneous recurrent convulsive seizures at the usual frequency of 2 to 5 per week (See, Turski et al., 1989; Synapse 3:154-171; Cavalheiro, 1995, Ital J Neurol Sci 16:33-37, Dube et al., 2001, Exp Neurol 167:227-241). The current antiepileptic drugs do not prevent the epileptogenesis and are only transiently efficient on recurrent seizures.

In our previous study, we studied the potential neuroprotective and antiepileptogenic effects of increasing doses of test compound given in monotherapy and compared to our standard diazepam treatment mostly given to prevent high mortality. These data show that a 7-day treatment with test compound at doses of 10, 30 or 60 mg/kg (RWJ10, RWJ 30, RWJ 60) starting at 1 h after the onset of SE is able to protect some brain areas from neuronal damage. This effect is statistically significant in the pyramidal cell layer of the CA1 and CA3b area, the mediodorsal thalamus, layers II and III-IV of the piriform cortex and layers III-IV of the ventral entorhinal cortex, but only at the highest dose of test compound, i.e. 60 mg/kg. (RWJ60) Moreover, it appears that the latter dose of test compound is also the only one that is able to delay the occurrence of spontaneous recurrent seizures (SRS), at least in a subgroup of animals that became epileptic with a mean delay that was about 9-fold longer than in the other groups of animals and one animal did not become epileptic in a delay of 9 months after SE.

In the present study, we tested the effects of different doses of various doses of test compound, i.e. 30, 60, 90 and 120 mg/kg (referred to herein as RWJ30, RWJ60, RWJ90 and RWJ120) using the same design as in the previous study. The treatment was started one hour after the onset of status epilepticus (SE) and the animals were treated with a second injection of the same dose of the drug. This early treatment of SE was followed by a 6 day test compound treatment. This report concerns the effects of the four different doses of test compound on neuronal damage in hippocampus and parahippocampal cortices. The study on the latency to the onset of epilepsy is still incomplete but we will show the data we have got at present which have been performed on a limited group of animals. The latency to spontaneous epileptic seizures was correlated on the same rats to the number of neurons remaining in the areas involved in the circuit of seizures.

Adult male Sprague-Dawley rats provided by Janvier Breeding Center (Le Genest-St-Isle, France) were housed under controlled, uncrowded standard conditions at 20-22° C. (light/dark cycle, 7.00 a.m.-7.00 p.m. lights on), with food and water available ad libitum. All animal experimentation was performed in accordance with the rules of the European Communities Council Directive of Nov. 24, 1986 (86/609/EEC), and the French Department of Agriculture (License No 67-97).

### Status Epilepticus Induction, Test Compound Treatment and Occurrence of SRS

All rats received lithium chloride (3 meq/kg, i.p., Sigma, St Louis, Mo., U.S.A.) and about 20 h later, all animals received also methylscopolamine bromide (1 mg/kg, s.c., Sigma) that was administered to limit the peripheral effects of the convulsant. SE was induced by injecting pilocarpine hydrochloride (25 mg/kg, s.c., Sigma) 30 min after methylscopolamine. The effects of increasing doses of test compound (RWJ) were studied in 5 groups of rats. The animals received either 2.5 mg/kg diazepam, i.m., (DZP), or 30, 60, 90 or 120 mg/kg of test compound (RWJ30, RWJ60, RWJ90, RWJ120), i.p., at 1 h after the onset of SE. The control group received vehicle instead of pilocarpine and test compound. The rats surviving SE were then injected about 10 h after the first test compound injection with a second i.p. injection of 1.25 mg/kg DZP for the DZP group or of the same dose of test compound as in the morning and were maintained under a twice daily test compound treatment for 6 additional days (s.c.) while DZP rats received a vehicle injection. The effects of the four doses of test compound on the latency to occurrence of spontaneous recurrent seizures (SRS) were investigated by daily video recording of the animals for 10 h per day.

### Quantification of Cell Densities

Quantification of cell densities was performed at two times after SE: a first group was studied 14 days after SE and was composed by 7 DZP, 8 RWJ30, 11 RWJ60, 10 RWJ90, 8 RWJ120 and 8 control rats not subjected to SE. A second group used for the study of the latency to SRS was sacrificed either 8 weeks after the first SRS or at 5 months when no SRS could be seen in that delay and was composed by 7 DZP, 2 RWJ30, 3 RWJ60, 3 RWJ90, 4 RWJ120 rats. Animals were deeply anesthetized with 1.8 g/kg pentobarbital (Dolethal®, Vétoquinol, Lure, France). Brains were then removed and frozen. Serial 20 μm slices were cut in a cryostat, air-dried during several days before thionine staining. Quantification of cell densities was performed with a 10×10 boxes 1 cm² microscopic grid on coronal sections according to the stereotaxic coordinates of the rat brain atlas (Paxinos and Watson, 1986, The Rat Brain in Stereotaxic coordinates, 2^{nd} ed. Academic Press, San Diego). The grid of counting was placed on a well-defined area of the cerebral structure of interest and counting was carried out with a microscopic enlargement of 200- or 400-fold defined for each single cerebral structure. Cell counts were performed twice on each side of three adjacent sections for each region by a single observer unaware of the animal's treatment. The number of cells obtained in the 12 counted fields in each cerebral structure was averaged. This procedure was used to minimize the potential errors that could result from double counting leading to overestimation of cell numbers. Neurons touching the inferior and right edges of the grid were not counted. Counts involved only neurons with cell bodies larger than 10 μm. Cells with small cell bodies were considered as glial cells and were not counted.

### Data Analysis

For neuronal damage, statistical analysis between groups was performed by means of a one-way analysis of variance followed by a post-hoc Scheffe's test using the Statview software.

### Results

### Behavioral Characteristics of Lithium-Pilocarpine Status Epilepticus

A total number of 101 Sprague-Dawley rats weighing 250-330 g were subjected to lithium-pilocarpine (Li-pilo)-induced SE. In this number 2 did not develop SE while 99 rats developed a full characteristic Li-pilo SE. The behavioral characteristics of SE were identical in both lipilo-DZP and lipilo-RWJ groups. Within 5 min after pilocarpine injection, rats developed diarrhea, piloerection and other signs of cholinergic stimulation. During the following 15-20 min, rats exhibited head bobbing, scratching, chewing and exploratory behavior. Recurrent seizures started around 15-20 min after pilocarpine administration. These seizures which associated episodes of head and bilateral forelimb myoclonus with rearing and falling progressed to SE at about 35-40 min after pilocarpine, as previously described (See: Turski et al., 1989; Synapse 3:154-171; Dubé et al., 2001, Exp Neurol 167:227-241 and André et al., 2003, Epilepsia 44:893-903).

In the group of animals devoted to early cell counting, and over the first 48 h after SE, the degree of mortality varied with the treatment: 47% (7/15) of DZP rats, 43% (6/14) of RWJ30 rats, 0% (0/11) of RWJ60 rats, 0% (0/10) of RWJ90 rats and 47% (7/15) of RWJ120 rats died. In the DZP group, 4/7 rats died in the first 24 h and 3 between 24 and 48 h. In the group of RWJ30 rats, 4/6 rats died during the first 24 h and 2 between 24 and 48 h. In the group of RWJ120 rats, 6/7 rats died during the first 24 h and 1 between 24 and 48 h. The control group not subjected to SE and receiving lithium and saline was composed of 10 rats.

In the group of animals devoted to the study of the latency to SRS and late cell counting, the degree of mortality was the following: 30% (3/10) of DZP rats, 50% (2/4) of RWJ30 rats, 0% (0/3) of RWJ60 rats, 0% (0/3) of RWJ90 rats and 0% (0/4) of RWJ120 rats died. In the group of DZP rats, 3/3 rats died during the first 24 h. In the group of RWJ30, ½ rats died during the first 24 h and 1 between 24 and 48 h.

### Cell Densities in Hippocampus and Cortex in the Early Phase (14 Days after SE)

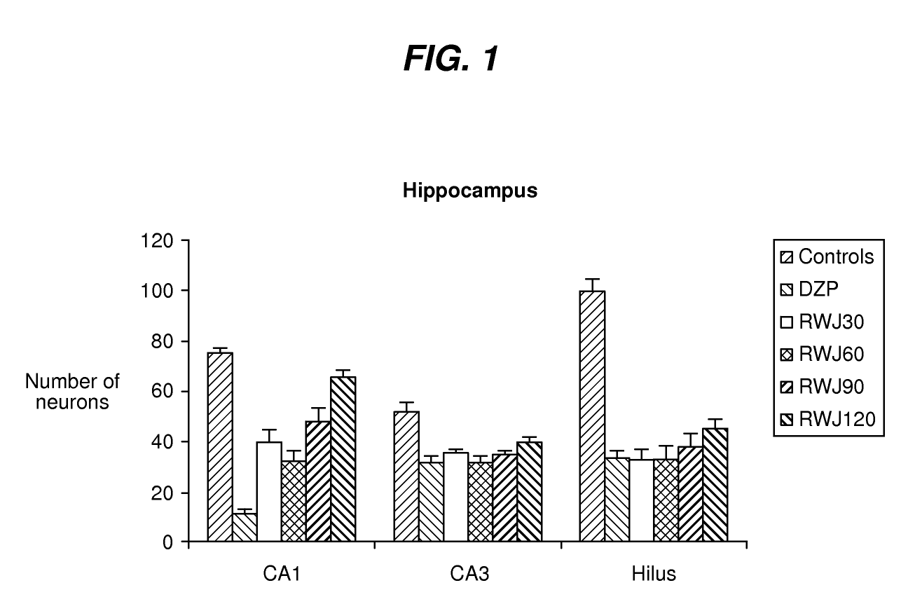

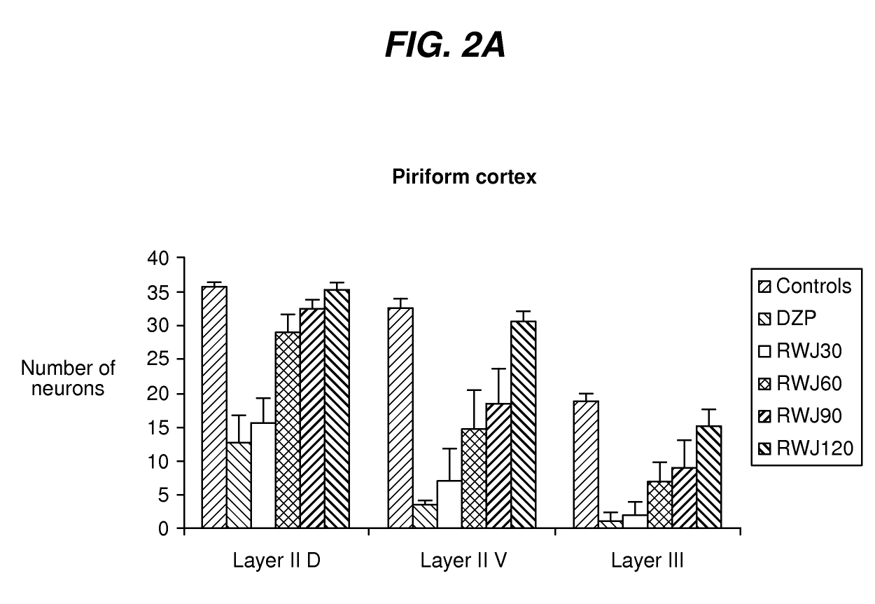

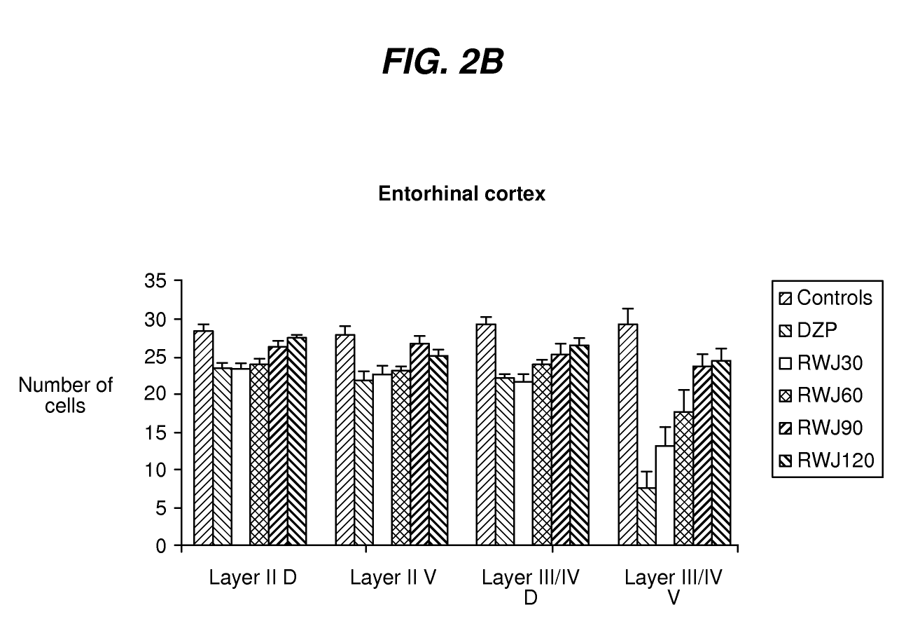

In DZP rats compared to control rats, the number of cells was massively decreased in the CA1 region of the hippocampus (85% cell loss in the pyramidal cell layer) while the CA3 region was less extensively damaged (40% cell loss in CA3) (See, Table 1 and FIG. 1). In the dentate gyrus, DZP rats experienced extensive cell loss in the hilus (66%) while the granule cell layer did usually not show visible damage. Similar damage was observed in the ventral hippocampus but cell counts were not performed in this region. In the piriform cortex, cell loss was almost total in layer III (94%) which was no longer really visible and reached 66 and 89% in dorsal and ventral layer II, respectively in DZP rats. In the dorsal entorhinal cortex, layers II and III-IV underwent slight damage (18 and 24%, respectively) and in ventral layers II and III/IV, damage reached 22 and 74%, respectively (Table 1 and FIG. 2).

In the hippocampus of RWJ animals, cell loss was somewhat reduced compared to DZP rats in the CA1 pyramidal layer (36-57% cell loss) in RWJ30, 60 or 90 rats and markedly reduced in the RWJ120 (13% cell loss). The differences were statistically significant at all RWJ doses (Table 1 and FIG. 1). In the CA3 pyramidal layer, there was a tendency to a slight neuroprotection induced by RWJ, only at the 120 mg/kg dose but the difference with the DZP group was not significant. In the dentate gyrus, the cell loss in the hilus was similar in the DZP and RWJ30, 60 and 90 groups (62-66% damage) and showed a slight tendency to reduced damage in the RWJ120 group (54% damage) compared to DZP animals (66% cell loss). None of these differences was statistically significant.

In the cerebral cortex, the treatment with test compound protected almost all cortical areas compared to the DZP treatment. In the piriform cortex, the RWJ30 dose did not protect any layer. Conversely, the 60-120 mg/kg doses of test compound afforded some neuroprotection in layer III (53-64% cell loss at the two lower doses and 21% cell loss at 120 mg/kg RWJ). The difference was only statistically significant at the highest dose. In dorsal layer II, 60-120 mg/kg induced a statistically significant neuroprotection (4-21% damage) compared to DZP-treated rats (66% damage). In ventral layer II, the 60 and 120 mg/kg RWJ dose were slightly protective (56 and 45% damage, respectively) while the 120 mg/kg largely protected this layer of the piriform cortex (9% damage) compared to DZP rats (89% damage). In all layers of the dorsal entorhinal cortex, the two lowest doses of test compound studied, 30 and 60 mg/kg did not afford any neuroprotection. The two highest doses of test compound tested, 90 and 120 mg/kg protected layers II and III/IV of the dorsal and ventral entorhinal cortex 4-14% damage remaining in layers II and II/IV of the dorsal part and in layer II of the ventral part compared to 18-24% in the DZP group and 18-21% cell losses in the ventral layers III/IV compared to 74% in the DZP group). However, this difference was statistically significant only in the ventral part of the entorhinal cortex.

### Cell Densities in the Piriform Cortex in the Early Phase (14 Days after SE)

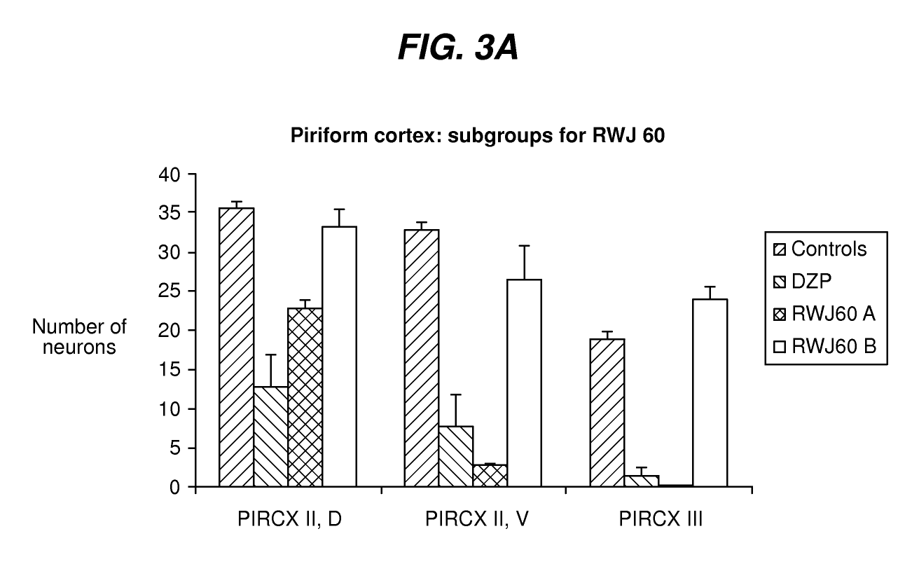

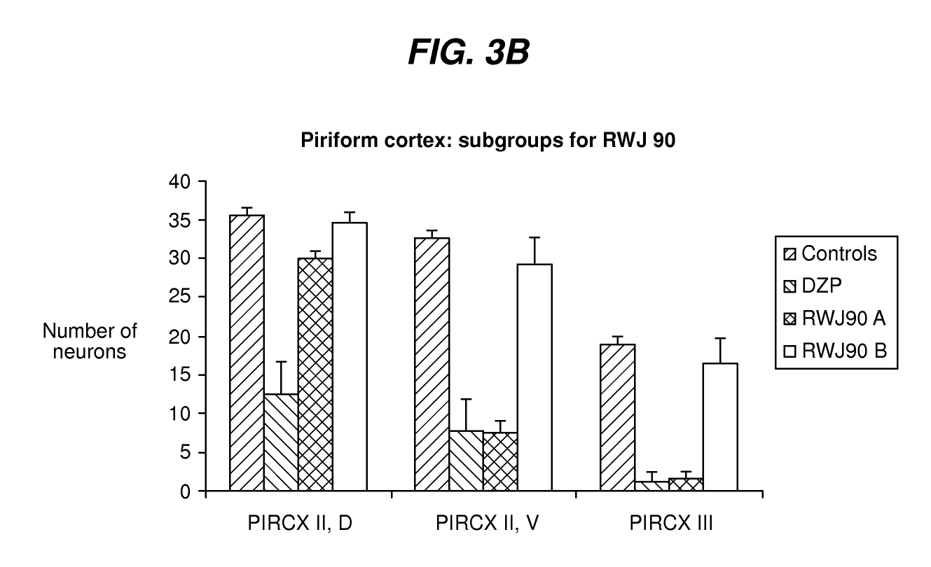

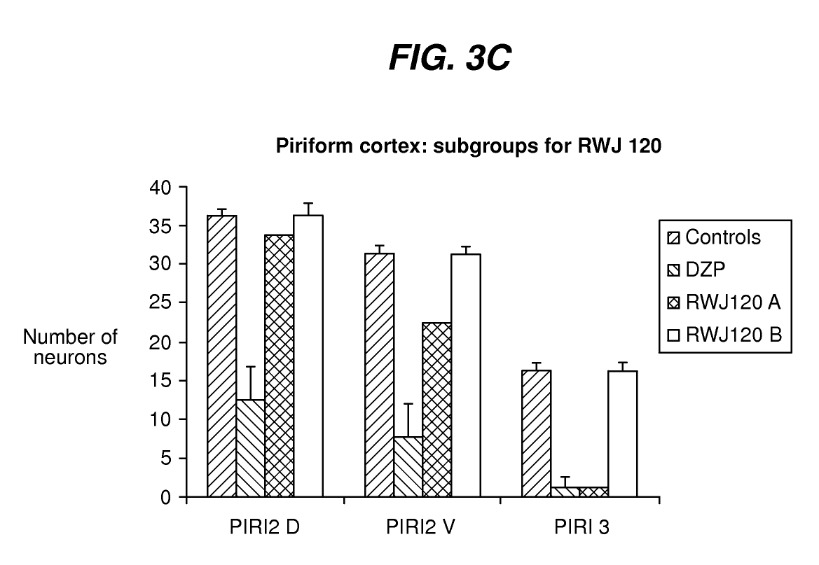

In the rats subjected to SE and treated with DZP or test compound at 30 mg/kg., the cell populations in the different areas of interest were in the same range in all animals. Conversely, at the three highest doses of test compound, two subgroups of rats could be distinguished according to the number of neurons surviving in the piriform cortex: one subgroup was almost totally protected while the other one was as extensively damaged as the DZP group. The differences between the number of neurons in the two subgroups were statistically significant in layer II of the ventral piriform cortex at the doses of 60 and 90 mg/kg test compound and in layer III of this same cortex in the three groups, test compound at 60, 90 and 120 mg/kg. (FIG. 3). There was a similar tendency in the entorhinal cortex but not as marked, probably because the damage is less extensive in this cortex and the difference never reached significance (data not shown).

### Latency to Recurrent Seizures

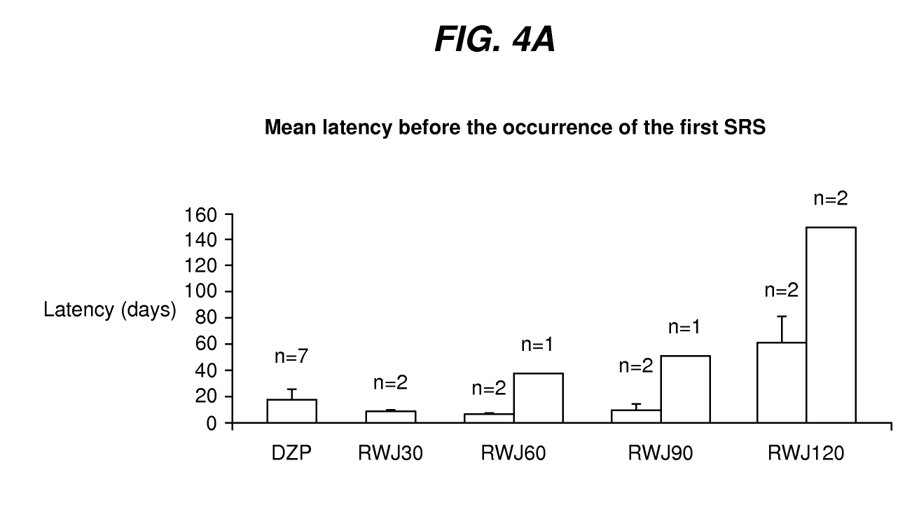

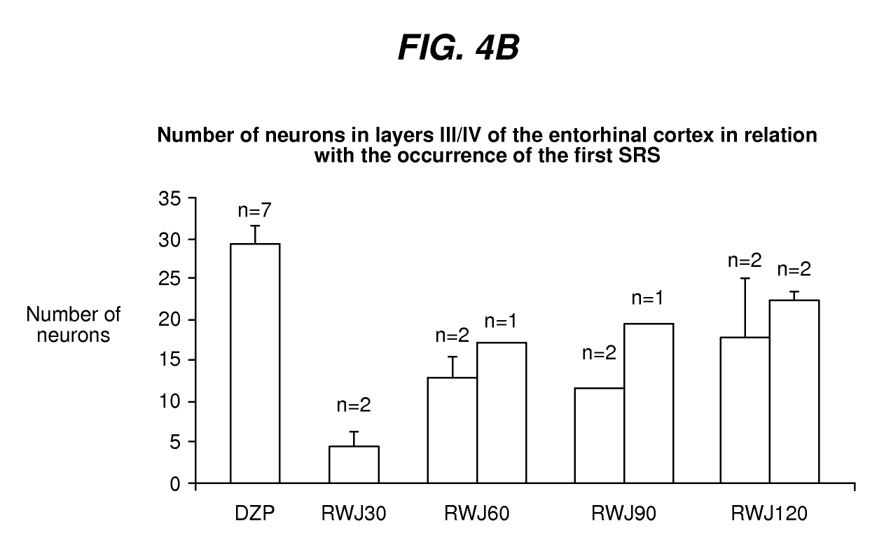

The only data that we have at this point concern the latencies in subgroups of rats in which we started the study on epileptogenesis. The latency to spontaneous seizures reached a mean value of 18±8 days in the DZP group (7 rats) and values of 8 and 9 days in the two rats of the test compound at 30 mg/kg. (RWJ30) group. At three other doses of DZP, there was a subdivision of the population with subgroups having a latency similar to the DZP and RWJ30 groups and subgroups with a prolonged latency to the first SRS. In the RWJ60 group, 2/3 rats had their first spontaneous seizure after 7 and 8 days, respectively while one rat exhibited its first seizure at 38 days after SE. At the 90 mg/kg test compound dose, 2/3 had their first spontaneous seizure after 9 and 10 days, respectively while one rat exhibited its first seizure at 51 days after SE. Finally, at the 120 mg/kg test compound dose, 2/4 rats had a prolonged latency and their first spontaneous seizure occurred after 44 and 79 days, respectively while the two other rats did not develop any seizure over the 5 months recording following SE (FIG. 4A).

### Correlation Between the Latency to Recurrent Seizures and the Number of Neurons Surviving in the Basal Cortices

When the number of neurons surviving in the cortical areas of the animals that have become epileptic after various latencies or have not become epileptic is plotted according to the latency to the first SRS, it seems that the duration of the latent period correlates with the number of neurons surviving in these areas. This is shown in FIG. 4B in layers III/IV of the ventral entorhinal cortex but seems to be the case also for layers III/IV of the dorsal entorhinal cortex and layer II of the piriform cortex. However, at the moment, the number of rats in each group is still much too low to allow us to draw a conclusion on a direct correlation between the percentage of neurons surviving SE after the test compound treatment in the ventral cortices and the antiepileptogenic effects of the drug.

### Discussion and Conclusions

The results of the present study show that a treatment with test compound starting at 1 h after the onset of Li-pilo-induced SE has neuroprotective properties in the CA1 pyramidal cell layer of the hippocampus, and in all layers of the piriform cortex and ventral and dorsal entorhinal cortex. However, in this model, test compound was not protective at the dose of 30 mg/kg and starts to display neuroprotective properties from the dose of 60 mg/kg. At the latter dose and at the two higher ones, 90 and 120 mg/kg, the drug is also able to delay the latency to the occurrence of the first spontaneous seizure but only in a subgroup of animals at each dose. In addition, this delay in the occurrence of epilepsy seems to correlate with the number of neurons surviving SE in the basal cortices in the same animals. However in this part of the study, the number of animals is still insufficient to firmly conclude on this correlation.

The data obtained in the present study are in line with the previous study from our group reporting that the 60 mg/kg dose of test compound protected the hippocampus and the basal cortices from neuronal damage and delayed the occurrence of recurrent seizures (see Example 1 above). They also confirm that the protection of the basal cortices seems to be a key factor in inducing a disease modifying effect in the lithium-pilocarpine model of epilepsy that is either the prolongation of the latent seizure-free phase or even its suppression at the 120 mg/kg. The key role of the basal cortices as initiators of the epileptic process was previously demonstrated by our group in the lithium-pilocarpine model (See, Andre et al., 2003; Epilepsia 44:893-903, Roch et al., 2002, Epilepsia 43:325-335 and Epilepsia 43:1129-1136).

In conclusion, test compound demonstrates very promising anti-epileptogenic and neuroprotective effects in this model and is to our knowledge the first molecule able to exhibit those properties. See Table 1 below;

TABLE 1
Effects of increasing doses of test compound on the number of
neuronal cell bodies in the hippocampus and cerebral cortex of rats subjected
to Li-Pilo SE. RWJ30 = 30 mg/kg test compound, RWJ60 = 60 mr/kg test
compound, RWJ90 = 90 mg/kg test compound and RWJ120 = 120 mg/kg test
compound.

| | Control | DZP | RWJ30 | RWJ60 | RWJ90 | RWJ120 |
|---|---|---|---|---|---|---|
| | (n = 10) | (n = 8) | (n = 8) | (n = 11) | (n = 10) | (n = 8) |
| Hippocampus | | | | | | |
| CA1 area | 74.9 ± 1.9° | 10.9 ± 1.9** | 39.3 ± 5.0°° | 31.9 ± 4.4 | 47.7 ± 5.4° | 65.5 ± 2.9° |
| CA3 area | 52.1 ± 2.7° | 31.3 ± 2.9** | 35.7 ± 1.4** | 31.6 ± 2.1** | 35.1 ± 1.0** | 39.8 ± 1.5** |
| Hilus | 97.9 ± 4.6° | 33.5 ± 3.0** | 33.0 ± 4.0** | 32.8 ± 5.4** | 37.5 ± 5.2**° | 44.8 ± 3.6* |
| Cerebral | | | | | | |
| cortex | | | | | | |
| Piriform, | 36.7 ± 0.9° | 12.6 ± 4.2** | 15.7 ± 3.6** | 28.9 ± 2.7°° | 32.4 ± 1.3° | 35.2 ± 1.3° |
| layer II, | | | | | | |
| dorsal | | | | | | |
| Piriform, | 33.5 ± 1.0° | 3.6 ± 0.7** | 7.2 ± 4.6** | 14.8 ± 5.9** | 18.4 ± 5.1 | 30.5 ± 1.5° |
| layer II, | | | | | | |
| ventral | | | | | | |
| Piriform, | 19.0 ± 0.9° | 1.2 ± 1.2** | 1.8 ± 2.3** | 6.9 ± 2.9 | 9.0 ± 4.1 | 15.0 ± 2.5°° |
| layer III | | | | | | |
| Dorsal | 28.6 ± 0.6° | 23.5 ± 0.7** | 23.4 ± 0.8** | 24.1 ± 0.7** | 26.3 ± 0.9 | 27.3 ± 0.5 |
| entorhinal, | | | | | | |
| layer II | | | | | | |
| Dorsal | 29.4 ± 1.1° | 22.3 ± 0.5** | 21.5 ± 1.3** | 24.0 ± 0.4** | 25.4 ± 1.3 | 26.4 ± 0.9 |
| entorhinal, | | | | | | |
| layer III | | | | | | |
| Ventral | 27.8 ± 0.9°° | 21.7 ± 1.3** | 22.7 ± 1.2* | 23.1 ± 0.7** | 26.7 ± 0.9°° | 25.1 ± 0.7 |
| entorhinal, | | | | | | |
| layer II | | | | | | |
| Ventral | 30.0 ± 2.3° | 7.7 ± 2.3** | 13.2 ± 2.5** | 17.8 ± 2.8* | 23.7 ± 1.7° | 24.5 ± 1.6° |
| entorhinal, | | | | | | |
| layers III-IV | | | | | | |

Values, expressed as the number of neuronal cell bodies in each area of interest, represent means ± S.E.M. of the number of animals in parentheses
*p < 0.05,
**p < 0.01, statistically significant differences from the control group
°p < 0.05,
°°p < 0.01, statistically significant difference from the DZP group

### Example 3

### PC12 Cell Serum Withdrawal Model

Serum withdrawal is a cytotoxic environmental challenge that results in cell death in cultured cell lines as well as in primary cells of various tissue origins, including nerve cells. In particular, pheochromocytoma (PC) 12 cells have been widely employed as an in vitro neuronal cell model for a wide variety of neurodegenerative and cell death related disorders (Muriel, et al, Mitochondrial free calcium levels (Rhod-2 fluorescence) and ultrastructural alterations in neuronally differentiated PC12 cells during ceramide-dependent cell death, J. Comp. Neurol., 2000, 426(2), 297-315; Dermitzaki, et al, Opioids transiently prevent activation of apoptotic mechanisms following short periods of serum withdrawal, J. Neurochem., 2000, 74(3), 960-969; Carlile, et al, Reduced apoptosis after nerve growth factor and serum withdrawal: conversion of tetrameric glyceraldehyde-3-phosphate dehydrogenase to a dimer, Mol. Pharmacol., 2000, 57(1), 2-12).

PC12 cells were cultured in sterile media (RPMI 1640) supplemented with 10% heat-inactivated horse serum and 5% fetal bovine serum (FBS). The culture medium also contained Penicillin-Streptomycin-Neomycin antibiotic (50.mu.g, 50.mu.g, 100.mu.g, respectively). Medium was exchanged every other day and the cells were passed in log phase near confluence.

The control cells were cultured in regular media without any treatment. An enantiomer of Formula 7 or Formula 8 (10.mu.M) was mixed well in the medium and then applied to the cells. For the 2 day assay, an enantiomer of Formula 7 or Formula 8 (10.mu.M) was only applied to the cells once at the time of serum withdrawal. For the 7 day assay, an enantiomer of Formula 7 or Formula 8 (10. mu.M) was applied to the cells at the time of serum withdrawal and every 48 hr thereafter when cells were changed with fresh new serum-free medium. In the serum withdrawal group, the cells were cultured in serum-free medium with no additional enantiomer of Formula 7 or Formula 8. Cell survival was determined by the 3-(4,5-dimethylthiazol-2-yl)-5-(3-carboxy-methoxyphenyl)-2-(4-sulfophenyl)-2H-tetrazolium inner salt (MTS) assay at 2 or 7 days after serum withdrawal.

At the end of the experiment, cells were washed with fresh medium and incubated with MTS solution in a humidified 37.degree. C. with 5% CO2 incubator for 1.5 hr. After the incubation period, the cells were immediately analyzed using a Softmax program (Molecular Devices). MTS assay is a calorimetric method for determining the number of viable cells in a given experimental setting. The assay is based on the cellular conversion of the tetrazolium salt, MTS, into a formazan that is soluble in tissue culture medium and measured directly at 490 nm in 96-well assay plates. The absorbance is directly proportional to the number of living cells in culture. The arbitrary absorbance reading in control cells is expressed as 100% survival rate. Table 2 lists data demonstrating the effect on cell survival rate of the orally administered enantiomer of Formula 7 and Formula 8 in the PC12 cell serum withdrawal model (.sup.1p value-0.01; .sup.2p value-<0.01).

TABLE 2
(% CELL SURVIVAL RATE)

| | 2 Day | 7 Day |
|---|---|---|
| | Survival Rate | Survival Rate |
| | (%) | (%) |
| Control | 100 | 100 |
| Serum-free | 49.6 ± 2.6 | 23.8 ± 2.6 |
| Formula 7 | 69.4 ± 1.7¹ | 79.9 ± 4.0² |
| Formula 8 | 66.4 ± 5.4¹ | 85.2 ± 0.6² |

### Example 4

### The Transient Cerebral Ischemia Rat Model

The enantiomer of Formula 7 was investigated in the transient cerebral ischemia middle cerebral artery occlusion (MCAO) rat model (as described in Nagasawa H. and Kogure K., Stroke, 1989, 20, 1037; and, Zea Longa E., Weinstein P. R., Carlson S, and Cummins R., Stroke, 1989, 20, 84) using male Wistar rats at 10 and 100 mg/kg (i.v.). MK 801 (Dizocilpine maleate; CAS Registry number 77086-22-7, a commercially available neuroprotectant compound) was used as a positive control (3 mg/kg, i.p.).

Rats (n=12) were randomly allocated to one of four experimental groups and were anesthetized. Blood flow from the internal carotid artery, anterior cerebral artery and posterior cerebral artery into the middle cerebral artery was blocked by this procedure. One hour after blockage, animals were treated over a 1 hour period with vehicle (administered i.v. over the one hour period), control (administered as a single i.p. dose at the start of the one hour period) and two doses of the enantiomer of Formula 7 (administered i.v. over the one hour period). Two hours after blockage, reperfusion was performed.

The animals were sacrificed and 20 mm-thick coronal sections of each brain were prepared. One in every forty sections (i.e. every 800 nM) from the front to the occipital cortex was used to quantify the extent of the cerebral lesion. Slides were prepared using sections stained (according to the Nissl procedure) with cresyl violet and were examined under a light microscope.

Regional ischemic surface areas in the coronal sections of individual rats were determined according to the presence of cells with morphological changes. The areas of neuronal injury or infarction were measured and then added. The cortex and striatum volume were calculated for each animal (total ischemic surface area.times.0.8 mm (thickness)).

### MCAO Model Analysis

The mean volumes (.+−.S.E.M.) for each animal randomly assigned to the four experimental groups were compared using one-way ANOVA (one way ANOVA is a statistical method which compares 3 or more unmatched groups) followed by Dunnett's t-test (both methods incorporated in Statview 512+software, BarinPower, Calabasas, Calif., USA).

As shown in Table 3 below, results were considered statistically significant when the p value was <0.05 compared to vehicle group (¹p<0.01; ²p<0.05).

TABLE 3

| | | Mean Infarct Volume (mm³) ± S.E.M. | | |
|---|---|---|---|---|
| | | Cortex | | |
| Treatment | N | Volume | Striatum | Total |
| Vehicle, 10 mL/kg | 12 | 275.5 ± 27.1 | 79.4 ± 3.6 | 354.9 ± 29.9 |
| MK 801 @ 3 mg/kg | 12 | 95.8 ± 24.5¹ | 56.1 ± 5.3² | 151.9 ± 28.7¹ |
| Formula 7 @ | 12 | 201.0 ± 23.9 | 75.9 ± 2.6 | 276.9 ± 25.4 |
| 10 mg/kg | | | | |
| Formula 7 @ | 12 | 98.8 ± 29.5¹ | 63.0 ± 5.9² | 161.9 ± 34.3¹ |
| 100 mg/kg | | | | |

### REFERENCES CITED

All references cited herein are incorporated herein by reference in their entirety and for all purposes to the same extent as if each individual publication or patent or patent application was specifically and individually indicated to be incorporated by reference in its entirety for all purposes.

The discussion of references herein is intended merely to summarize the assertions made by their authors and no admission is made that any reference constitutes prior art. Applicants reserve the right to challenge the accuracy and pertinence of the cited references.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

### REFERENCES

Dublin W B (1976) Fundamentals of sensorineural auditory pathology. Springfield, Ill.: C. C. Thomas.
Rybak L P (1986) Ototoxic mechanisms. In: Neurobiology of Hearing. Altschuler R A, Bobbin R P, Hoffman D W, Eds. Raven Press (New York) PP 441-454.
Lim D J (1986) Effects of noise and ototoxic drugs at the cellular level in the cochlea: A review. Am J Otolaryngol 7: 73-99.
Pryor G (1994) Assessment of auditory dysfunction. In Principle of Neurotoxicology. Chang L W, ed., Marcel Dekker, Inc. PP 345-371.
Fleischman, R. W., Stadnicki, S. W., Ethier, M. F., and Schaeppi, U. (1975). Ototoxicity of cis-dichlorodiammine platinum (II) in the guinea pig. Toxicol Appl. Pharmacol. 33:320-332.
Stadnicki, S. W., Fleischman, R. W., Schaeppi, U., and Merriam, P. (1975). Cis-dichlorodiammineplatinum (II) (NSC-119875): Hearing loss and other toxic effects in rhesus monkeys. Cancer Chemother. Rep. 59:467-480.
Nakai, Y., Konishi, K., Chang, K. C., Ohashi, K., Morisaki, N., Minowa, Y., and Morimoto, A. (1982). Ototoxicity of the anticancer drug cisplatin. Acta Otolaryngol 93:227-232.
Berggren, D, Anniko, M, Thornell, L.-E., Ramaekers, F. C. S., and Virtanem, I. (1990). Intermediate filament proteins in the embryonic inner ear of mice under normal conditions and after exposure to ototoxic drugs. Acta Otolaryngol. (Stockh) 109:57-65.
Hood J L, Berlin C I, Ed., Contemporary applications of neurobiology in human hearing assessment (Raven Press, New York, 1986).
Sera, K., Harada, Y., Tagashira, N., Suzuki, M., Hirakawa, K., and Ohya, T. (1987). Morphological changes in the vestibular epithelia and ganglion induced by ototoxic drug. Scanning Microsc. 1:1191-1197.
Hinojosa, R., and Lerner, S. A. (1987). Cochlear neural degeration without hair cell loss in two patients with aminoglycoside ototoxicity. J. Infect Dis. 156: 449-455
Bareggi, R., Grill, V., Narducci, P., Zweyer, M., Tesei, L, and Russolo, M. (1990). Genetamicin ototoxicity: Histological and ulstructural alterations after transtympanic administration. Pharmacol. Res. 22:635-644.
Carenza, L., Villani, C., Framarino dei Malatesta, M. L., Prosperi Porta, R., Millefiorine, M., Antonini, G., Bolasco, P., Bandiera, G., and Marzetti, L. (1986). Peripheral neuropathy and ototoxicity of dichlorodiamineplatinum: instrumental evaluation. Gynecol. Oncol. 25:244-249.
Myers E N, Berstaein J M (1965) Salicylate ototoxicity: A clinical and experimental study. Arch Otolaryngl Head Neck Surg 82: 483-493.
Jarvis J F (1966) A case of unilateral permanent deafness following acetyl salicylic acid. J Laryngol 80: 318-320.
Rahman, M U et al. Curr. Opin. Rheumatol., May; 13(3):184-9, (2001))
Choi et al., (1988), Neuron 1: 623-634;
Rothman et al., (1984), J. Neurosci. 4: 1884-1891;
Choi end Rothman, (1990), Ann. Rev. Neurosci. 13: 171-182;
David et al., (1988), Exp. Eye Res. 46:657-662;
Drejer et al., (1985), J. Neurosci. 45:145-151.
Reagan, R. F., Excitotoxicity and Central Nervous System Trauma, in The Neurobiology of Central Nervous Trauma, New York, Oxford University Press, 1994, pp. 173-181 (Salzman S K, Faden A I, eds)
Robinson, M. B. & Dowd L A, Adv Pharmacol, 1997; 37:69-115
Kermer et al., Cell Tissue Res 298:383-395, 1999
Coyle and Schwartz, (1976), Nature 263: 244-246;
Alzheimer's disease (AD) (Maragos et al, (1987), TINS 10: 65-68;
Epilepsy (Nadler et al, (1978), Nature 271: 676-677)
Lathyrism (Spencer et al, (1986), Lancet 239: 1066-1067; 5)
Caine et al, (1986), Lancet 2: 1067-1070
Dykens et al, (1987), J. Neurochem. 49: 1222-1228).
Lipton et al. (1994) New Engl. J. Med. 330:613 621)
Meldrum et al. (1990) Trends Pharm. Sci. 11:379-387).
Herin et al. (2001) J. Neurochem. 78:1307-1314
Rossato et al. (2002) Neurosci. Lett. 318:137-140).
Jacques, et al., Enantiomers, Racemates and Resolutions (Wiley Interscience, New York, 1981);
Wilen, S. H., et al., Tetrahedron 33:2725 (1977);
Eliel, E. L. Stereochemistry of Carbon Compounds (McGraw-Hill, NY, 1962);
Wilen, S. H. Tables of Resolving Agents and Optical Resolutions p. 268 (E. L. Eliel, Ed., Univ. of Notre Dame Press, Notre Dame, Ind. 1972).
Alfonso A R: Remington's Pharmaceutical Sciences, 17th ed., Mack Publishing Company, Easton Pa., 1985
Minto, J. Pharmacol. Exp. Ther. 281:93-102, 1997.
Rohatagi, J. Clin. Pharmacol. 35:1187-1193, 1995; Tjwa, Ann. Allergy Asthma Immunol. 75:107-111, 1995).
Rao, J. Biomater Sci. Polym. Ed. 7:623-645, 1995;
Gao, Pharm. Res. 12:857-863, 1995);
Eyles, J. Pharm. Pharmacol. 49:669-674, 1997)
Al-Muhammed, J. Microencapsul. 13:293-306, 1996;
Chonn, Curr. Opin. Biotechnol. 6:698-708, 1995;
Ostro, Am. J. Hosp. Pharm. 46:1576-1587, 1989).
Pharmaceutical Dosage Forms: Tablets. Second Edition. Revised and Expanded. Volumes 1-3, edited by Lieberman et al;
Pharmaceutical Dosage Forms: Parenteral Medications. Volumes 1-2, edited by Avis et al;
Pharmaceutical Dosage Forms: Disperse Systems. Volumes 1-2, edited by
Lieberman et al; published by Marcel Dekker, Inc,
Lieberman, Pharmaceutical Dosage Forms (Vols. 1-3, 1992); Lloyd, 1999,
The art, Science and Technology of Pharmaceutical Compounding; and Pickar, 1999, Dosage Calculations
Turski et al., 1989, Synapse 3:154-171;
Cavalheiro, 1995, Ital J Neurol Sci 16:33-37)
Dube et al., 2001, Exp Neurol 167:227-241).
Paxinos and Watson, 1986, The Rat brain in Stereotaxic Coordinates, 2^{nd} ed. Academic Press, San Diego)
Cavazos et al., 1991, J Neurosci 11:2795-2803.)
Fisher R A, 1946a, Statistical Methods for Research Workers (10th edition) Oliver & Boyd, Edinburgh
Fisher R A, 1946b, The Design of Experiments (4th edition) Oliver & Boyd, Edinburgh)
Turski et al., 1983, Behav Brain Res 9:315-335.
André et al., 2003, Epilepsia 44:893-903
Roch et al., 2002, Epilepsia 43:325-335
Roch et al., 2002, Epilepsia 43:1129-1136
Muriel, et al, Mitochondrial free calcium levels (Rhod-2 fluorescence) and ultrastructural alterations in neuronally differentiated PC12 cells during ceramide-dependent cell death, J. Comp. Neurol., 2000, 426(2), 297-315
Dermitzaki, et al, Opioids transiently prevent activation of apoptotic mechanisms following short periods of serum withdrawal, J. Neurochem., 2000, 74(3), 960-969;
Carlile, et al, Reduced apoptosis after nerve growth factor and serum withdrawal: conversion of tetrameric glyceraldehyde-3-phosphate dehydrogenase to a dimer, Mol. Pharmacol., 2000, 57(1), 2-12).
Nagasawa H. and Kogure K., Stroke, 1989, 20, 1037;
Zea Longa E., Weinstein P. R., Carlson S, and Cummins R., Stroke, 1989, 20, 84)

1. A method for providing otoprotection, comprising administering to a patient in need of treatment with a otoprotective drug (an OPD) a therapeutically effective amount of a compound, or a pharmaceutically acceptable salt or ester thereof, selected from the group consisting of Formula (I) and Formula (II): 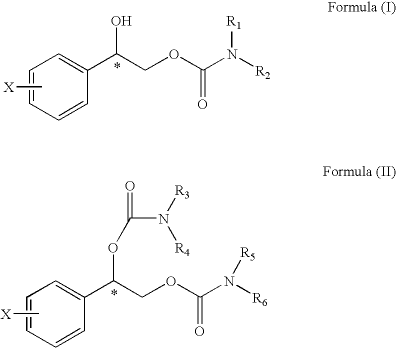
wherein
phenyl is substituted at X with one to five halogen atoms selected from the group consisting of fluorine, chlorine, bromine and iodine; and, R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

2. The method of claim 1 wherein X is chlorine substituted at the ortho position of the phenyl ring and wherein R₁, R₂, R₃, R₄, R₅ and R₆ are selected from hydrogen.

3. A method for providing otoprotection, comprising administering to a patient in need of treatment with a otoprotective drug (an OPD) a therapeutically effective amount of an enantiomer, or a pharmaceutically acceptable salt or ester thereof, selected from the group consisting of Formula (I) and Formula (II) or enantiomeric mixture wherein one enantiomer selected from the group consisting of Formula (I) and Formula (II) predominates: 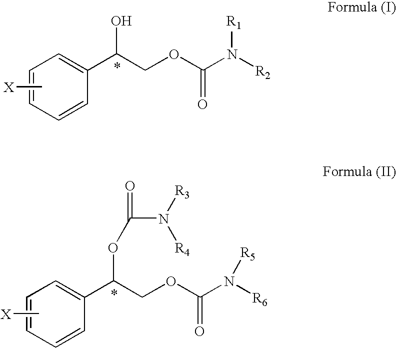
wherein
phenyl is substituted at X with one to five halogen atoms selected from the group consisting of fluorine, chlorine, bromine and iodine; and, R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

4. The method of claim 3 wherein X is chlorine substituted at the ortho position of the phenyl ring and wherein R₁, R₂, R₃, R₄, R₅ and R₆ are selected from hydrogen.

5. The method of claim 3 wherein one enantiomer selected from the group consisting of Formula (I) and Formula (II) predominates to the extent of about 90% or greater.

6. The method of claim 3 wherein the enantiomer selected from the group consisting of Formula (I) and Formula (II) is an enantiomer selected from the group consisting of Formula (Ia) and Formula (IIa): 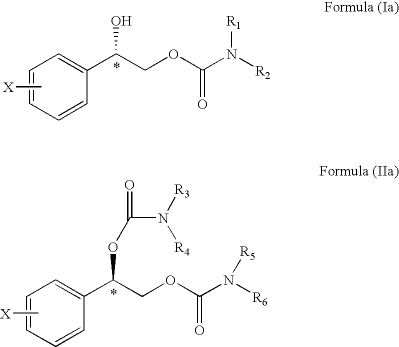
wherein
phenyl is substituted at X with one to five halogen atoms selected from the group consisting of fluorine, chlorine, bromine and iodine; and, R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

7. The method of claim 6 wherein X is chlorine substituted at the ortho position of the phenyl ring and wherein R₁, R₂, R₃, R₄, R₅ and R₆ are selected from hydrogen.

8. The method of claim 6 wherein one enantiomer selected from the group consisting of Formula (Ia) and Formula (IIa) predominates to the extent of about 90% or greater.

9. The method of claim 3 wherein the enantiomer selected from the group consisting of Formula (I) and Formula (II) is an enantiomer selected from the group consisting of Formula (Ib) and Formula (IIb):

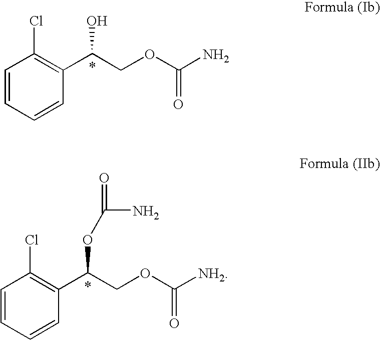

10. The method of claim 9 wherein one enantiomer selected from the group consisting of Formula (Ib) and Formula (IIb) predominates to the extent of about 90% or greater.

11. The method, as claimed in claims 1 or 3 wherein the possible cause(s) of otic damage rendering the patient in need of otoprotection are selected from the group consisting of: a condition selected from; ototoxic drug exposure, trauma, tinnitus, Meniere's Disease including immune-mediated Meniere's disease, immune-mediated cochlear or vestibular disorders (IMCVD), autoimmune ear disease (AIED), Cogan's Syndrome, ischemic events affecting the inner ear or eight nerve and blunt or penetrating head trauma.

12. The method of claim 11 wherein the predisposing factor(s) rendering the patients in need of otoprotection are selected from the group consisting: tinnitus, Meniere's Disease and ototoxic drug exposure.

13. The method of claim 12 wherein the said predisposing factor is tinnitus.

14. The method of claim 12 wherein the said predisposing factor(s) are ototoxic drug exposure.

15. The method of claim 14 wherein the said ototoxic drug is an antibiotic.

16. The method of claim 14 wherein the said ototoxic drug is a chemotherapy agent.

17. The methods of claims 1 or 3 wherein said compound (or enantiomer) or a pharmaceutically acceptable salt or ester thereof is administered in combination administration with one or more other compounds or therapeutic agents.

18. The methods of claim 17 wherein the said one or more other compounds or therapeutic agents are selected from the group consisting of antibiotics and chemotherapy agents; such that ototoxicity is inhibited in the patient.

19. A pharmaceutical composition for providing otoprotection comprising a pharmaceutically effective amount of an enantiomer, or a pharmaceutically acceptable salt or ester thereof, selected from the group consisting of Formula (Ib) and Formula (IIb) or an enantiomeric mixture wherein one enantiomer selected from the group consisting of Formula (Ib) and Formula (IIb):

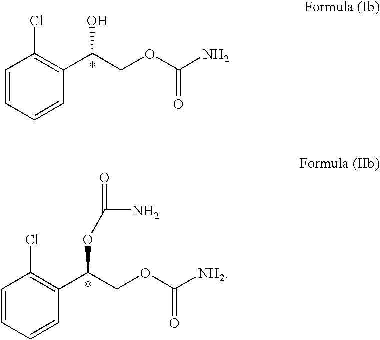

20. The method as in claims 1 or 3 wherein the therapeutically effective amount is from about 0.1 mg/kg/dose to about 50 mg/kg/dose.

21. The method as in claims 1 or 3 wherein the therapeutically effective amount is from about 1.0/mg/kg/dose to about 25 mg/kg/dose,

22. The method as in claims 1 or 3 wherein the therapeutically effective amount is from about 2.0 to about 40 mg/kg/dose.

23. The method as in claims 1 or 3 wherein the therapeutically effective amount is from about 140 mg/day to about 2800 mg/day for a subject having an average weight of 70 kg.
